(19) United States
(12) Patent Application Publication
(10) Pub. No.: US 2016/0215119 A1
(43) Pub. Date: Jul. 28, 2016

(54) TETRAHYDROFURAN DERIVATIVES AND THEIR USE AS PLASTICIZERS

(71) Applicant: BASF SE, Ludwigshafen (DE)

(72) Inventors: JOCHEN WAGNER, Ruppertsweiler (DE); Boris BREITSCHEIDEL, Waldsee (DE); Martin A. BOHN, Mannheim (DE); Benoit BLANK, Edingen-Neckarhausen (DE); Alois KINDLER, Grünstadt (DE)

(21) Appl. No.: 14/916,230

(22) PCT Filed: Sep. 3, 2014
(86) PCT No.: PCT/EP2014/068687

§ 371 Date: Mar. 3, 2016

(30) Foreign Application Priority Data
Sep. 4, 2013 (EP) 13182979.8

(51) Int. Cl. C08K 5/1535; C08L 27/06; C08K 5/00; C07D 307/24; C07D 307/12

(52) U.S. Cl. CPC C08K 5/1535; C07D 307/24; C07D 307/12; C08K 5/0016; C08L 27/06

(57) ABSTRACT

The present invention relates to tetrahydrofuran derivatives, a plasticizer composition containing said tetrahydrofuran derivatives, molding materials containing a thermoplastic polymer and such a tetrahydrofuran derivative, to a process for the production of these tetrahydrofuran derivatives and their use.

### CROSS-REFERENCE TO RELATED APPLICATIONS

This application is a national stage application (under 35 U.S.C. §371) of PCT/EP2014/068687, filed Sep. 3, 2014, which claims benefit of European Application No. 13182979.8, filed Sep. 4, 2013, both of which are incorporated herein by reference in their entirety.

### BACKGROUND OF THE INVENTION

The present invention relates to tetrahydrofuran derivatives, to a plasticizer composition which comprises said tetrahydrofuran derivatives, to molding compositions which comprise a thermoplastic polymer and a tetrahydrofuran derivative of this type, to a process for producing said tetrahydrofuran derivatives, and to use of these.

### PRIOR ART

Desired processing properties or desired performance characteristics are achieved in many plastics by adding what are known as plasticizers in order to render the plastics softer, more flexible and/or more extensible. Plasticizers generally serve to shift the thermoplastic region of plastics to lower temperatures, so as to obtain the desired elastic properties at lower processing temperatures and lower usage temperatures.

Production quantities of polyvinyl chloride (PVC) are among the highest of any plastic. Because this material is versatile, it is nowadays found in a wide variety of products used in everyday life. PVC therefore has very great economic importance. PVC is intrinsically a plastic that is hard and brittle up to about 80° C., and is used in the form of rigid PVC (PVC-U) by adding heat stabilizers and other additives. Flexible PVC (PVC-P) is obtained only by adding suitable plasticizers, and can be used for many applications for which rigid PVC is unsuitable.

Examples of other important thermoplastic polymers in which plasticizers are usually used are polyvinyl butyral (PVB), homo- and copolymers of styrene, polyacrylates, polysulfides, and thermoplastic polyurethanes (PUs).

There are many different compounds marketed for plasticizing PVC and other plastics. Phthalic diesters with alcohols of different chemical structure have in the past often been used as plasticizers because they have good compatibility with PVC and advantageous performance characteristics, examples being diethylhexyl phthalate (DEHP), diisononyl phthalate (DINP) and diisodecyl phthalate (DIDP). Short-chain phthalates, e.g. dibutyl phthalate (DBP), diisobutyl phthalate (DIBP), benzyl butyl phthalate (BBP) or diisoheptyl phthalate (DIHP), are also used as gelling aids (“fast fuser”), for example in the production of what are known as plastisols. It is also possible to use dibenzoic esters, such as dipropylene glycol dibenzoates, for the same purpose alongside the short-chain phthalates. Phenyl esters of alkylsulfonic acids are another class of plasticizers with good gelling properties, and are marketed by way of example in the form of mixtures as Mesamoll® TP-LXS 51067.

In particular in the production and processing of flexible PVC and of PVC plastisols, for example for producing PVC foils or PVC coatings, it is inter alia desirable to have available a plasticizer with minimal gelling point and low viscosity. High storage stability of the plasticizer/plastic mixtures is moreover also desirable, i.e. the latter in their ungelled form are intended to exhibit no, or only a slight, viscosity rise over the course of time at ambient temperature. As far as possible, these properties are intended to be achieved by addition of a suitable plasticizer with good gelling properties, with no need for the use of other viscosity-reducing additives and/or of solvents.

Another known method for establishing the desired properties is to use mixtures of plasticizers, e.g. to use at least one plasticizer which provides good thermoplastic properties but has poor gelling effect, in combination with at least one plasticizer which has good gelling properties.

There is a need to replace the phthalate plasticizers mentioned in the introduction, because these are not entirely free from toxicological concerns. This specifically applies to sensitive application sectors such as toys, food packaging, or medical items.

Various alternate plasticizers for a variety of plastics, and specifically for PVC, are known in the prior art.

A plasticizer class that is known from the prior art and that can be used as alternative to phthalates is based on the cyclohexanepolycarboxylic acids described in WO 99/32427. Unlike their unhydrogenated aromatic analogs, these compounds give rise to no toxicological concerns, and can be used even in sensitive application sectors. This plasticizer class includes inter alia the diisononyl esters of 1,2-cyclohexanedicarboxylic acid, which are marketed for example by BASF SE as isomer mixture with trademark Hexamoll® DINCH® (CAS No. in Europe and Asia: 166412-78-8; CAS No. in the USA: 474919-59-0) and which are widely used as plasticizers for various polymers.

WO 00/78704 describes selected dialkylcyclohexane-1,3- and 1,4-dicarboxylic esters for the use as plasticizer in synthetic materials.

U.S. Pat. No. 7,973,194 B1 teaches the use of dibenzyl cyclohexane-1,4-dicarboxylate, benzyl butyl cyclohexane-1,4-dicarboxylate, and dibutyl cyclohexane-1,4-dicarboxylate as rapid-gelling plasticizers for PVC.

Some diether derivatives of 2,5-di(hydroxymethyl)tetrahydrofuran are already known materials. WO 2009/141166 describes a fuel composition composed of ring-hydrogenated alkylfurfuryl ethers of the general formula: R″-TF-CH₂—O—R, in which TF is a 2,5-disubstituted tetrahydrofuran ring, R is a hydrocarbyl group having from 1 to 20 carbon atoms, R″ represents a methyl group, a hydroxymethyl group, or else the product of an aldol condensation, or represents an alkoxymethyl group of the general formula: —CH₂—O—R′, in which R′ is a hydrocarbyl group having from 1 to 20 carbon atoms. Only methyl and ethyl are specifically used as moiety R and R′. Said document claims that these compounds are novel materials, and also describes a process for producing these, but teaches only use of these as fuel or fuel additives, rather than as plasticizer.

The esters of 2,5-furandicarboxylic acid (FDCA) are another plasticizer class.

WO 2012/113608 describes C₅-dialkyl esters of 2,5-furandicarboxylic acid and use of these as plasticizers. These short-chain esters are specifically also suitable for producing plastisols.

WO 2012/113609 describes C₇-dialkyl esters of 2,5-furandicarboxylic acid and use of these as plasticizers.

WO 2011/023490 describes C₉-dialkyl esters of 2,5-furandicarboxylic acid and use of these as plasticizers.

WO 2011/023491 describes C₁₀-dialkyl esters of 2,5-furandicarboxylic acid and use of these as plasticizers.

R. D. Sanderson et al. (J. Appl. Pol. Sci., 1994, vol. 53, 1785-1793) describe the synthesis of esters of 2,5-furandicarboxylic acid and use of these as plasticizers for plastics, in particular polyvinyl chloride (PVC), polyvinyl butyral (PVB), polylactic acid (PLA), polyhydroxybutyric acid (PHB) or polyalkyl methacrylate (PAMA). Specifically, the di(2-ethylhexyl), di(2-octyl), dihexyl, and dibutyl esters of 2,5-furandicarboxylic acid are described, and the plasticizing properties of these are characterized by way of dynamic mechanical thermal analyses.

U.S. Pat. No. 3,259,636 describes a process for producing esters of cis-2,5-tetrahydrofurandicarboxylic acid, where hydrogen, 2,5-furandicarboxylic acid and an alcohol are reacted in the presence of a noble metal catalyst in a one-pot reaction. Specifically, production of the methyl, propyl and phenoxyethyl diesters of cis-2,5-tetrahydrofurandicarboxylic acid is described. It is moreover disclosed that the esters of alcohols having 6 or more carbon atoms are suitable as plasticizers in resin compositions.

Another important field of application for plasticizers is production of what are known as plastisols. Plastisols initially are a suspension of finely pulverulent plastics in liquid plasticizers. The solvation rate of the polymer in the plasticizer here is very low at ambient temperature. The polymer is noticeably solvated in the plasticizer only on heating to relatively high temperatures. The individual isolated polymer aggregates here swell and fuse to give a three-dimensional high-viscosity gel. This procedure is termed gelling, and begins at a certain minimum temperature which is termed gel point or solvation temperature. The gelling step is not reversible.

Since plastisols take the form of liquids, these are very often used for the coating of a very wide variety of materials, e.g. textiles, glass nonwovens, etc. This coating is very often composed of a plurality of sublayers.

In a procedure often used in the industrial processing of plastisols, a layer of plastisol is therefore applied and then the plastic, in particular PVC, with the plasticizer is subjected to incipient gelling above the solvation temperature, thus producing a solid layer composed of a mixture of gelled, partially gelled, and ungelled polymer particles.

The next sublayer is then applied to this incipiently gelled layer, and once the final layer has been applied the entire structure is processed in its entirety to give the fully gelled plastics product by heating to relatively high temperatures.

Another possibility, alongside production of plastisols, is production of dry pulverulent mixtures of plasticizer and polymers. These dry blends, in particular based on PVC, can then be further processed at elevated temperatures for example by extrusion to give pellets, or processed through conventional shaping processes, such as injection molding, extrusion, or calendering, to give the fully gelled plastics product.

It is an object of the present invention to provide novel compounds which can advantageously be used as, or in, plasticizers for thermoplastic polymers and elastomers. They are intended to be free from toxicological concerns and to be capable of production from readily obtainable starting materials which preferably at least to some extent derive from renewable raw materials. They are intended to have good plasticizing properties and therefore to permit production of products with good mechanical properties, such as low Shore hardness, low cold crack temperature, or high ultimate tensile strength. The compounds are also intended to have good gelling properties and/or to have low viscosity in the ungelled state, and therefore to be suitable in particular for the production of flexible PVC and of PVC plastisols. The novel compounds should accordingly be capable of providing an at least equivalent replacement for the standard petrochemically based plasticizers that are mainly used nowadays.

Surprisingly, said object is achieved via tetrahydrofuran derivatives of the general formula (I)

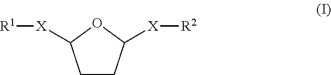

in which

X is *—(C═O)—O—, *—(CH₂)ₙ—O— or *—(CH₂)ₙ—O—(C═O)—, where * is the point of linkage to the tetrahydrofuran ring, and n has the value 0, 1, or 2;

and

R¹ and R² are selected mutually independently from unbranched and branched O₇-O₁₂-alkyl moieties.

The invention further provides plasticizer compositions which comprise at least one compound of the general formula (I) as defined above and hereinafter, and at least one plasticizer different from the compounds of the formula (I).

The invention further provides processes for producing compounds of the general formula (I).

The invention further provides the use of compounds of the general formula (I) as, or in, plasticizers for polymers, in particular for polyvinyl chloride (PVC).

The invention further provides molding compositions which comprise at least one thermoplastic polymer and at least one compound of the general formula (I) as defined above and hereinafter.

The invention further provides molding compositions which comprise at least one elastomer and at least one compound of the general formula (I) as defined above and hereinafter.

The invention further provides the use of said molding compositions for producing moldings and foils.

### DESCRIPTION OF FIGURES

FIG. 1 shows, in the form of a bar chart, the Shore A hardness of flexible PVC test specimens which comprise different amounts of the plasticizer 2,5-THFDCA di(2-propylheptyl) ester (white hatched) and, as comparison, the commercially available plasticizer Hexamoll® DINCH® (black). The Shore A hardness has been plotted against the plasticizer content of the flexible PVC test specimens (stated in phr). The values measured were always determined after a time of 15 seconds.

FIG. 2 shows, in the form of a bar chart, the Shore D hardness of flexible PVC test specimens which comprise 50 and, respectively, 70 phr of the plasticizer 2,5-THFDCA di(2-propylheptyl) ester of the invention (white hatched) and, as comparison, the commercially available plasticizer Hexarnoll® DINCH® (black). The Shore D hardness has been plotted against the plasticizer content of the flexible PVC test specimens (stated in phr). The values measured were always determined after a time of 15 seconds.

FIG. 3 shows, in the form of a bar chart, the 100% modulus of flexible PVC test specimens which comprise 50 and, respectively, 70 phr of the plasticizer 2,5-THFDCA di(2-propylheptyl) ester of the invention (white hatched) and, as comparison, the commercially available plasticizer Hexamoll® DINCH® (black). The 100% modulus has been plotted against the plasticizer content of the flexible PVC test specimens (stated in phr).

FIG. 4 shows, in the form of a bar chart, the cold crack temperature of flexible PVC foils which comprise the plasticizer 2,5-THFDCA di(2-propylheptyl) ester of the invention and, as comparison, the commercially available plasticizer Hexamoll® DINCH®. The chart shows the cold crack temperature in ° C. for flexible PVC foils with plasticizer content of 50 and 70 phr.

FIG. 5 shows, in the form of a bar chart, the glass transition temperature (T_{g}) of flexible PVC foils which comprise the plasticizer 2,5-THFDCA di(2-propylheptyl) ester of the invention and, as comparison, the commercially available plasticizer Hexamoll® DINCH®. The chart shows the glass transition temperature (T_{g}) in ° C. for flexible PVC foils with plasticizer content of 50 and 70 phr.

FIG. 6 shows, in the form of a bar chart, the ultimate tensile strength of flexible PVC foils which comprise the plasticizer 2,5-THFDCA di(2-propylheptyl) ester of the invention and, as comparison, the commercially available plasticizer Hexamoll® DINCH®. The chart shows the ultimate tensile strength in MPa for flexible PVC foils with plasticizer content of 50 and 70 phr.

FIG. 7 shows, in the form of a bar chart, the tensile strain at break of flexible PVC foils which comprise the plasticizer 2,5-THFDCA di(2-propylheptyl) ester of the invention and, as comparison, the commercially available plasticizer Hexamoll® DINCH®. The chart shows the tensile strain at break in % of the initial value (=100%) for flexible PVC foils with plasticizer content of 50 and 70 phr.

FIG. 8 shows the gelling behavior of PVC plastisols which comprise the plasticizer 2,5-THFDCA di(2-propylheptyl) ester of the invention and, as comparison, the commercially available plasticizer Hexamoll® DINCH®. The viscosity of the plastisols is shown as a function of temperature.

### DESCRIPTION OF THE INVENTION

The compounds (I) of the invention exhibit the following advantages:
By virtue of their physical properties, the compounds (I) of the invention have very good suitability for applications as plasticizers or as component of a plasticizer composition for thermoplastic polymers, in particular for PVC.
The polymers plasticized with the compounds (I) of the invention have good mechanical properties, such as low Shore hardness or high ultimate tensile strength.
By virtue of their low solvation temperatures in accordance with DIN 53408, the compounds (I) of the invention have very good gelling properties. They are therefore suitable for reducing the temperature required for gelling of a thermoplastic polymer and/or for increasing the gelling rate.
The compounds of the general formula (I) of the invention feature very good compatibility with a wide variety of different plasticizers. They are specifically suitable in combination with conventional plasticizers for improving gelling performance.
The compounds (I) of the invention are advantageously suitable for producing plastisols.
The compounds (I) of the invention are suitable for the use for producing moldings and foils for sensitive application sectors, for example medical products, food packaging, products for the interior sector, for example in dwellings and in vehicles, and for toys, child-care items, etc.
The compounds (I) of the invention can be produced by using readily obtainable starting materials. A particular economic and environmental advantage of the present invention derives from the possibility of using, in the production of the compounds (I) of the invention, not only petrochemical raw materials that are available in large quantities but also renewable raw materials. By way of example, therefore, it is possible to obtain the starting materials for the furan rings from naturally occurring carbohydrates, such as cellulose and starch, while the alcohols that can be used for introducing the side chains are available from large-scale industrial processes. It is thus possible on the one hand to comply with the “sustainable” materials requirement while on the other hand also permitting cost-effective production.
The processes for producing the compounds (I) of the invention are simple and efficient, and these can therefore be provided without difficulty on a large industrial scale.

As previously mentioned, it has surprisingly been found that the compounds of the general formula (I), in particular the C₇-C₁₂-dialkyl esters of tetrahydrofurandicarboxylic acid, have very good suitability for plasticizing thermoplastic polymers, and permit production of products with good mechanical properties. Surprisingly, it has also been found that these compounds have low solvation temperatures, and also excellent gelling properties in the production of flexible PVC and of plastisols, in particular of PVC plastisols: their solvation temperatures are below the solvation temperatures of the corresponding dialkyl esters of 2,5-furandicarboxylic acid or phthalic acid, and they have at least equivalent gelling properties. This was not to be expected, since by way of example ring-hydrogenated phthalates such as diisononyl cyclohexane-1,2-dicarboxylate generally have higher solvation temperatures than their unhydrogenated forms: by way of example, the solvation temperature of diisononyl 1,2-cyclohexanedicarboxylate is higher at 151° C. than that of diisononyl phthalate at 132° C., in accordance with DIN 53408.

The compounds of the general formula (I.1) of the invention can take the form either of pure cis-isomers or of pure trans-isomers, or of cis/trans-isomer mixtures. The pure isomers and the isomer mixtures of any desired composition are equally suitable as plasticizers.

For the purposes of the present invention, the expression “C₁-C₃-alkyl” comprises straight-chain or branched C₁-C₃-alkyl groups. Among these are methyl, ethyl, propyl, and isopropyl. Methyl is particularly preferred.

The expression “C₇-C₁₂-alkyl” comprises straight-chain and branched C₇-C₁₂-alkyl groups. It is preferable that C₇-C₁₂-alkyl is selected from n-heptyl, 1-methylhexyl, 2-methylhexyl, 1-ethylpentyl, 2-ethylpentyl, 1-propylbutyl, 1-ethyl-2-methylpropyl, n-octyl, isooctyl, 2-ethylhexyl, n-nonyl, isononyl, 2-propylhexyl, n-decyl, isodecyl, 2-propylheptyl, n-undecyl, isoundecyl, n-dodecyl, isododecyl, and the like. It is particularly preferable that C₇-C₁₂-alkyl is n-octyl, n-nonyl, isononyl, 2-ethylhexyl, isodecyl, 2-propylheptyl, n-undecyl, or isoundecyl.

It is preferable that the definitions of the groups X in the compounds of the general formula (I) are identical.

In a first preferred embodiment, both of the groups X in the compounds of the general formula (I) are *—(C═O)—O—.

In another preferred embodiment, both of the groups X in the compounds of the general formula (I) are *—(CH₂)—O—(C═O)—.

In another preferred embodiment, both of the groups X in the compounds of the general formula (I) are *—(CH₂)ₙ—O—, where n is 0, 1 or 2. It is particularly preferable that n is 2.

It is preferable that the moieties R¹ and R² in the compounds of the general formula (I) are mutually independently an unbranched or branched C₇-C₁₂-alkyl moiety.

It is particularly preferable that the moieties R¹ and R² in the compounds of the general formula (I) are mutually independently isononyl, 2-propylheptyl, or 2-ethylhexyl.

In a preferred embodiment, the definitions of the moieties R¹ and R² in the compounds of the general formula (I) are identical.

Preferred compounds of the general formula (I) are those selected from
di(isononyl) 2,5-tetrahydrofurandicarboxylate,
di(2-propylheptyl) 2,5-tetrahydrofurandicarboxylate,
di(2-ethylhexyl) 2,5-tetrahydrofurandicarboxylate,
diisononyl ether of 2,5-di(hydroxymethyl)tetrahydrofuran,
di-2-propylheptyl ether of 2,5-di(hydroxymethyl)tetrahydrofuran,
di-2-ethylhexyl ether of 2,5-di(hydroxymethyl)tetrahydrofuran,
2,5-di(hydroxymethyl)tetrahydrofuran diisononanoate,
2,5-di(hydroxymethyl)tetrahydrofuran di-2-propylheptanoate,
2,5-di(hydroxymethyl)tetrahydrofuran di-2-ethylheptanoate,

and also mixtures of 2 or more of the abovementioned compounds.

A particularly preferred compound of the general formula (I) is di(2-propylheptyl) 2,5-tetrahydrofurandicarboxylate.

Another particularly preferred compound of the general formula (I) is di(isononyl) 2,5-tetrahydrofurandicarboxylate.

Another particularly preferred compound of the general formula (I) is di(2-ethylhexyl) 2,5-tetrahydrofurandicarboxylate.

Production of the Compounds of the General Formula (I)

Production of the diesters of 2,5-tetrahydrofurandicarboxylic acid

The invention further provides a process for producing compounds of the general formula (I.1),

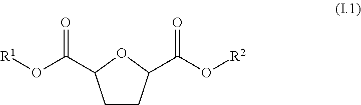

in which
R¹ and R² are selected mutually independently from branched and unbranched C₇-C₁₂-alkyl moieties,

where
a) optionally 2,5-furandicarboxylic acid or an anhydride or acyl halide thereof is reacted with a C₁-C₃-alkanol in the presence of a catalyst to give a di(C₁-C₃-alkyl) 2,5-furandicarboxylate,
b1) 2,5-furandicarboxylic acid or an anhydride or acyl halide thereof, or the di(C₁-C₃-alkyl) 2,5-furandicarboxylate obtained in step a), is reacted with at least one alcohol R¹—OH and, if R¹ and R² are different, also with at least one alcohol R²—OH, in the presence of at least one catalyst to give a compound of the formula (I.1a),

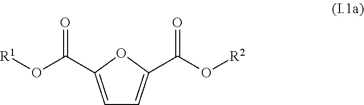

c1) the compound (I.1a) obtained in step b1) is hydrogenated with hydrogen in the presence of at least one hydrogenation catalyst to give the compound of the general formula (I.1),

or
b2) 2,5-furandicarboxylic acid or the di(C₁-C₃-alkyl) 2,5-furandicarboxylate obtained in step a) is hydrogenated with hydrogen in the presence of at least one hydrogenation catalyst to give a compound of the general formula (I.1b),

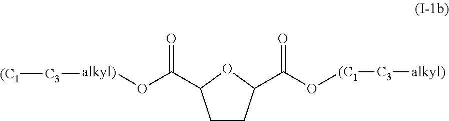

c2) the compound (I.1 b) obtained in step b2) is reacted with at least one alcohol R¹—OH and, if R¹ and R² are different, also with at least one alcohol R²—OH, in the presence of a catalyst to give a compound of the formula (I.1).

In respect of suitable and preferred embodiments of the moieties R¹ and R², reference is made to the entirety of the information provided above.

The process of the invention permits the production of the 2,5-tetrahydrofurandicarboxylic esters of the general formula (I.1) by two different routes (hereinafter termed variant 1 and variant 2).

Examples of C₁-C₃-alkanols suitable for use in step a) are methanol, ethanol, n-propanol, and mixtures thereof.

In variant 1 of the process of the invention, the 2,5-furandicarboxylic acid or the di(C₁-C₃-alkyl) 2,5-furandicarboxylate obtained in step a) is subjected to esterification or transesterification with at least one alcohol R¹—OH and, if R¹ and R² are different, also with at least one alcohol R²—OH, to give the compounds of the formula (I.1a), which are then hydrogenated to give compounds of the general formula (I.1) (step c1)).

In variant 2, the 2,5-furandicarboxylic acid or the 2,5-di(C₁-C₃-alkyl) furandicarboxylate obtained in step a) is first hydrogenated to give 2,5-tetrahydrofurandicarboxylic acid or, respectively, a compound of the general formula (I.1b) (step b2)), and the hydrogenation product is then reacted with at least one alcohol R¹—OH and, if R¹ and R² are different, also with at least one alcohol R²—OH to give the compounds of the general formula (I.1) (step c2)).

Esterification

Conventional processes known to the person skilled in the art can be used to convert the 2,5-furandicarboxylic acid (FDCA) or the 2,5-tetrahydrofurandicarboxylic acid to the corresponding ester compounds of the general formulae (I.1), (I.1a), and (I.1b). Among these are the reaction of at least one alcohol component selected from C₁-C₃-alkanols or from the alcohols R¹—OH and, respectively, R²—OH with FDCA or a suitable derivative thereof. Examples of suitable derivatives are the acyl halides and anhydrides. A preferred acyl halide is the acyl chloride. Esterification catalysts that can be used are the catalysts conventionally used for this purpose, e.g. mineral acids, such as sulfuric acid and phosphoric acid; organic sulfonic acids, such as methanesulfonic acid and p-toluenesulfonic acid; amphoteric catalysts, in particular titanium compounds, tin(IV) compounds, or zirconium compounds, e.g. tetraalkoxytitanium compounds, e.g. tetrabutoxytitanium, and tin(IV) oxide. The water produced during the reaction can be removed by conventional measures, e.g. by distillation. WO 02/038531 describes a process for producing esters where a) a mixture consisting essentially of the acid component or an anhydride thereof and of the alcohol component is heated to boiling point in the presence of an esterification catalyst in a reaction zone, b) the vapors comprising alcohol and water are fractionated to give an alcohol-rich fraction and a water-rich fraction, c) the alcohol-rich fraction is returned to the reaction zone, and the water-rich fraction is discharged from the process. Esterification catalysts used are the abovementioned catalysts. An effective amount of the esterification catalyst is used and is usually in the range from 0.05 to 10% by weight, preferably from 0.1 to 5% by weight, based on the entirety of acid component (or anhydride) and alcohol component. Other detailed descriptions of the conduct of esterification processes are found by way of example in U.S. Pat. No. 6,310,235, U.S. Pat. No. 5,324,853, DE-A 2612355 (Derwent Abstract No. DW 77-72638 Y) or DE-A 1945359 (Derwent Abstract No. DW 73-27151 U). The entirety of the documents mentioned is incorporated herein by way of reference.

In one preferred embodiment, the esterification of FDCA or of the 2,5-tetrahydrofurandicarboxylic acid is carried out in the presence of the alcohol components described above by means of an organic acid or mineral acid, in particular concentrated sulfuric acid. The amount used of the alcohol component here is advantageously at least twice the stochiometric amount, based on the FDCA or the 2,5-tetrahydrofurandicarboxylic acid or a derivative.

The esterification can generally take place at ambient pressure or at reduced or elevated pressure. It is preferable that the esterification is carried out at ambient pressure or reduced pressure.

The esterification can be carried out in the absence of any added solvent or in the presence of an organic solvent.

If the esterification is carried out in the presence of a solvent, it is preferable that the organic solvent used is inert under the reaction conditions. Among these are by way of example aliphatic hydrocarbons, halogenated aliphatic hydrocarbons, and aromatic and substituted aromatic hydrocarbons and ethers. It is preferable that the solvent is one selected from pentane, hexane, heptane, ligroin, petrol ether, cyclohexane, dichloromethane, trichloromethane, tetrachloromethane, benzene, toluene, xylene, chlorobenzene, dichlorobenzenes, dibutyl ether, THF, dioxane, and mixtures thereof.

The esterification is usually carried out in the temperature range from 50 to 250° C.

If the esterification catalyst is one selected from organic acids or mineral acids, the esterification is usually carried out in the temperature range from 50 to 160° C.

If the esterification catalyst is one selected from amphoteric catalysts, the esterification is usually carried out in the temperature range from 100 to 250° C.

The esterification can take place in the absence of or in the presence of an inert gas. The expression inert gas generally means a gas which under the prevailing reaction conditions does not enter into any reactions with the starting materials, reagents, or solvents participating in the reaction, or with the resultant products. It is preferable that the esterification takes place without addition of any inert gas.

Transesterification:

Conventional processes known to the person skilled in the art can be used for the reaction, described in steps b1) and c2), of the di(C₁-C₃-alkyl) 2,5-furandicarboxylates and, respectively, the di(C₁-C₃-alkyl) 2,5-tetrahydrofurandicarboxylates to give the corresponding ester compounds 1.1a and, respectively, 1.1. Among these are the reaction of the di(C₁-C₃)-alkyl esters with at least one C₇-C₁₂-alkanol or a mixture thereof in the presence of a suitable transesterification catalyst.

Transesterification catalysts that can be used are the conventional catalysts usually used for transesterification reactions, where these are mostly also used in esterification reactions. Among these are by way of example mineral acids, such as sulfuric acid and phosphoric acid; organic sulfonic acids, such as methanesulfonic acid and p-toluenesulfonic acid; and specific metal catalysts from the group of the tin(IV) catalysts, for example dialkyltin dicarboxylates, such as dibutyltin diacetate, trialkyltin alkoxides, monoalkyltin compounds, such as monobutyltin dioxide, tin salts, such as tin acetate, or tin oxides; from the group of the titanium catalysts: monomeric and polymeric titanates and titanium chelates, for example tetraethyl orthotitanate, tetrapropyl orthotitanate, tetrabutyl orthotitanate, triethanolamine titanate; from the group of the zirconium catalysts: zirconates and zirconium chelates, for example tetrapropyl zirconate, tetrabutyl zirconate, triethanolamine zirconate; and also lithium catalysts, such as lithium salts, lithium alkoxides; and aluminum(III) acetylacetonate, chromium(III) acetylacetonate, iron(III) acetylacetonate, cobalt(II) acetylacetonate, nickel(II) acetylacetonate, and zinc(II) acetylacetonate.

The amount of transesterification catalyst used is from 0.001 to 10% by weight, preferably from 0.05 to 5% by weight. The reaction mixture is preferably heated to the boiling point of the reaction mixture, the reaction temperature therefore being from 20° C. to 200° C., depending on the reactants.

The transesterification can take place at ambient pressure or at reduced or elevated pressure. It is preferable that the transesterification is carried out at a pressure of from 0.001 to 200 bar, particularly from 0.01 to 5 bar. The relatively low-boiling-point alcohol eliminated during the transesterification is preferably continuously removed by distillation in order to shift the equilibrium of the transesterification reaction. The distillation column necessary for this purpose generally has direct connection to the transesterification reactor, and it is preferable that said column is a direct attachment thereto. If a plurality of transesterification reactors are used in series, each of said reactors can have a distillation column, or the vaporized alcohol mixture can preferably be introduced into a distillation column from the final tanks of the transesterification reactor cascade by way of one or more collection lines. The relatively high-boiling-point alcohol reclaimed in said distillation is preferably returned to the transesterification.

If an amphoteric catalyst is used, this is generally successfully removed via hydrolysis and subsequent removal of the metal oxide formed, for example by filtration. It is preferable that, once the reaction has taken place, the catalyst is hydrolyzed by washing with water, and that the precipitated metal oxide is removed by filtration. If desired, the filtrate may be subjected to further workup for isolation and/or purification of the product. The product is preferably isolated by distillation.

In one preferred embodiment of steps 1b) and 2c), the transesterification of the di(C₁-C₃-alkyl) 2,5-furandicarboxylates and, respectively, di(C₁-C₃-alkyl) 2,5-tetrahydrofurandicarboxylates takes place in the presence of the alcohol component and in the presence of at least one titanium(IV) alcoholate. Preferred titanium(IV) alcoholates are tetrapropoxytitanium, tetrabutoxytitanium, and mixtures thereof. It is preferable that the amount used of the alcohol component is at least twice the stochiometric amount, based on the di(C₁-C₃-alkyl) ester used.

The transesterification can be carried out in the absence of, or in the presence of, an added organic solvent. It is preferable that the transesterification is carried out in the presence of an inert organic solvent. Suitable organic solvents are those mentioned above for the esterification. Among these are specifically toluene and THF.

The transesterification is preferably carried out in the temperature range from 50 to 200° C.

The transesterification can take place in the absence of or in the presence of an inert gas. The expression inert gas generally means a gas which under the prevailing reaction conditions does not enter into any reactions with the starting materials, reagents, or solvents participating in the reaction, or with the resultant products. It is preferable that the transesterification takes place without addition of any inert gas.

Hydrogenation

Many processes and catalysts for the hydrogenation of the double bonds of the furan ring carried out in steps c1) and b2) of the invention are available to the person skilled in the art and these by way of example are also used in the hydrogenation of esters of aromatic polycarboxylic acids, examples being phthalates, isophthalates and terephthalates. By way of example, the ring-hydrogenation process described in WO 99/032427 is suitable. This comprises hydrogenation at from 50 to 250° C. and at a pressure of from 20 to 300 bar by means of catalysts which comprise at least one metal of transition group VIII of the Periodic Table of the Elements, for example platinum, rhodium, palladium, cobalt, nickel, or ruthenium, preferably ruthenium, either alone or together with at least one metal from transition group I or VII of the Periodic Table of the Elements, for example copper or ruthenium, deposited on a mesoporous aluminum oxide support material with bimodal pore distribution. The ring-hydrogenation process described in WO 02/100536 is moreover suitable. This comprises hydrogenation with use of a ruthenium catalyst on amorphous silicon dioxide as support. Other suitable processes are described in the following documents: EP-A 1266882—Use of a nickel/magnesium oxide on kieselguhr catalyst, WO 03/029181—Use of a nickel/zinc on silicon dioxide catalyst, WO 03/029168—Use of a palladium/ZnO on Al₂O₃ catalyst and of a ruthenium/ZnO on α-Al₂O₃ catalyst, or WO 04/09526—Use of a ruthenium on titanium dioxide catalyst. Other suitable catalysts are likewise Raney catalysts, preferably Raney nickel. Other suitable support materials alongside those already mentioned are by way of example zirconium dioxide (ZrO₂), sulfated zirconium dioxide, tungsten carbide (WC), titanium dioxide (TiO₂), sulfated carbon, activated charcoal, aluminum phosphate, aluminosilicates, or phosphated aluminum oxide, or else a combination thereof.

The hydrogenation can take place by analogy with the known hydrogenation processes for hydrogenating organic compounds which have hydrogenatable groups. To this end, the organic compound in the form of liquid phase or gas phase, preferably in the form of liquid phase, is brought into contact with the catalyst in the presence of hydrogen. The liquid phase can by way of example be passed over a fluidized bed of catalyst (fluidized bed method) or can be passed over a fixed bed of catalyst (fixed bed method).

In the process of the invention, it is preferable that the hydrogenation takes place in a fixed-bed reactor.

The hydrogenation can be designed to take place either continuously or else batchwise, preference being given here to the continuous design of the process. The batchwise hydrogenation can use a reaction apparatus conventionally used for this purpose, e.g. a stirred reactor. It is preferable that the hydrogenation of the invention is carried out continuously in fixed-bed reactors in upflow mode or downflow mode. The hydrogen here can be passed over the catalyst cocurrently with the solution of the starting material to be hydrogenated, or else in countercurrent.

Suitable apparatuses for conducting fluidized-bed-catalyst hydrogenation and fixed-bed-catalyst hydrogenation are known in the prior art, e.g. from Ullmanns Enzyklopadie der Technischen Chemie [Ullmann's Encyclopedia of Industrial Chemistry], 4^{th} edition, volume 13, pp. 135 ff., and also from P. N. Rylander, “Hydrogenation and Dehydrogenation” in Ullmann's Encyclopedia of Industrial Chemistry, 5th edn. on CDROM.

The hydrogenation generally takes place under elevated hydrogen pressure. Preference is given to hydrogen pressure in the range from 2 to 500 bar, particularly from 10 to 300 bar.

It is preferable that the hydrogenation takes place in the presence of an organic solvent that is inert under the hydrogenation conditions. Suitable solvents are those previously defined for the esterification. Specifically, an ether is used, for example THF, or a dialkylene glycol, or a mono- or diether thereof, for example glyme.

The hydrogenation is carried out at a temperature in the range from 20 to 350° C., particularly preferably from 50 to 300° C.

The amount of hydrogen used for the hydrogenation is generally from 1 to 15 times the stochiometric amount of hydrogen theoretically needed for the complete hydrogenation of the furan ring.

In one preferred embodiment of steps c1) and b2), the hydrogenation of the furan ring is carried out with platinum, rhodium, palladium, cobalt, nickel, or ruthenium, in particular platinum and palladium, deposited on aluminum oxide, on zirconium dioxide, on sulfated zirconium dioxide, on zinc oxide, or on silicon dioxide, in particular on zirconium dioxide, in the presence of an inert solvent, under hydrogen pressure of from 150 to 300 bar, at a temperature of from 150 to 250° C.

The hydrogenation processes described can give preference to formation of the cis- or trans-isomer of the 2,5-tetrahydrofurandicarboxylic esters in accordance with the selected hydrogenation conditions, for example catalyst composition, or hydrogenation temperature: it is possible to produce cis- or trans-2,5-tetrahydrofurandicarboxylic esters that are in essence isomerically pure, or else a mixture with various proportions of cis- and trans-isomers. The expression “in essence isomerically pure” here means content of at least 95% by weight of a particular isomer, based on the total weight of the 2,5-tetrahydrofurandicarboxylic ester.

The compounds of the general formula (I.1) of the invention can accordingly take the form of pure cis-isomers or take the form of pure trans-isomers, or take the form of cis/trans-isomer mixtures. The pure isomers and the isomer mixtures of any desired composition are equally suitable as plasticizers.

In one particularly preferred embodiment of steps c1) and b2), FDCA and, respectively, the esters of the 2,5-furandicarboxylic acid from steps a) and b1) are dissolved in an inert solvent and fully hydrogenated in the presence of a heterogeneous Pd/Pt catalyst at a hydrogen pressure of from 50 to 300 bar and at from 100 to 250° C. The hydrogenation here preferably takes place continuously by the fixed-bed method, where the hydrogen is conducted in countercurrent over the catalyst. In this embodiment, it is preferable to use THF as solvent. In this embodiment, it is moreover preferable to use a Pd/Pt catalyst on ZrO₂. The preferred reaction temperature for this embodiment is in the range from 100 to 200° C. In this embodiment, the desired tetrahydrofuran derivatives are generally obtained with a proportion of cis-isomer of at least 90% by weight, based on the total amount of the cis/trans-isomers formed.

One particularly preferred embodiment of the process of the invention comprises:
a) reaction of 2,5-furandicarboxylic acid with methanol in the presence of concentrated sulfuric acid to give the dimethyl 2,5-furandicarboxylate,
2b) hydrogenation of the dimethyl 2,5-furandicarboxylate obtained in step a) with hydrogen in the presence of a Pd/Pt catalyst on ZrO₂ to give the dimethyl 2,5-tetrahydrofurandicarboxylate,
2c) reaction of the dimethyl 2,5-tetrahydrofurandicarboxylate obtained in step 2b) with at least one alcohol R¹—OH in the presence of at least one titanium(IV) alcoholate to give the compounds of the general formula (I.1).

Production of the C₇-C₁₂-diether derivatives and, respectively, C₇-C₁₂-diester derivatives of the formulae (I.2) and, respectively, (I.3)

The invention further provides a process for producing compounds of the general formula (I.2) or (I.3),

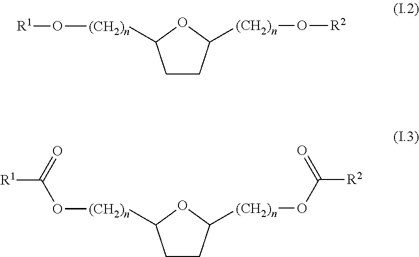

in which

R¹ and R² are selected mutually independently from unbranched and branched C₇-C₁₂-alkyl moieties, and n has the value 1 or 2,

where
for 2,5-di(hydroxymethyl)tetrahydrofuran (n=1) or for 2,5-di(hydroxyethyl)tetrahydrofuran (n=2), reaction is carried out with at least one alkylating reagent R¹—Z and, if R¹ and R² are different, also with at least one alkylating reagent R²—Z, where Z is a leaving group, in the presence of a base to give compounds of the formula (I.2),

or
for 2,5-di(hydroxymethyl)tetrahydrofuran (n=1) or for 2,5-di(hydroxyethyl)tetrahydrofuran (n=2), reaction is carried out with at least one acyl halide R¹—(C═O)X and, if R¹ and R² are different, also with at least one acyl halide R²—(C═O)X, where X is Br or Cl, in the presence of at least one tertiary amine to give compounds of the formula (I.3).

The alkylation is generally carried out in the presence of an organic solvent that is inert under the reaction conditions. Suitable solvents are those previously mentioned for the estification. Aromatic hydrocarbons, such as toluene, are preferred as solvent.

The leaving group Z is preferably a moiety selected from Br, Cl, and the tosyl, mesyl, and triflyl group.

It is particularly preferable that the leaving group Z is Br.

The alkylation reagents R¹—Z and R²—Z are available commercially or can be produced by way of suitable reactions or procedures familiar to the person skilled in the art, from the corresponding alcohols. By way of example, the alkyl bromides R¹—Br and, respectively, R²—Br preferably used for this process can be produced in a known manner on a large industrial scale from the appropriate alcohols R¹—OH and, respectively, R²—OH by using hydrogen bromide (HBr).

Suitable bases that can be used in the process of the invention are mineral bases and/or strong organic bases. Among these are by way of example inorganic bases or base-formers, for example hydroxides, hydrides, amides, oxides, and carbonates of the alkali metals and of the alkaline earth metals. Among these are LiOH, NaOH, KOH, Mg(OH)₂, Ca(OH)₂, LiH, NaH, sodium amide (NaNH₂), lithium diisopropylamide (LDA), Na₂O, K₂CO₃, Na₂CO₃, and Cs₂CO₃; and also organometallic compounds, such as nBuLi, or tert-BuLi. Preference is given to NaOH, KOH, K₂CO₃, and Na₂CO₃.

The amount used here of the base is preferably at least a two-fold stoichiometric excess, based on the 2,5-di(hydroxymethyl)tetrahydrofuran and, respectively, 2,5-di(hydroxyethyl)tetrahydrofuran. It is particularly preferable to use an at least four-fold stoichiometric excess of base.

The alkylation can be carried out in the absence of, or in the presence of, an organic solvent. The reaction is generally carried out in the presence of an inert organic solvent, such as pentane, hexane, heptane, ligroin, petroleum ether, cyclohexane, dichloromethane, trichloromethane, tetrachloromethane, benzene, toluene, xylene, chlorobenzene, dichlorobenzenes, dibutyl ether, THF, dioxane, or a mixture thereof.

The alkylation can generally take place at ambient pressure, reduced pressure, or elevated pressure. It is preferable that the alkylation is carried out at ambient pressure.

It is preferable that the alkylation is carried out in the temperature range from 30 to 200° C., preferably from 50 to 150° C.

The alkylation can take place in the absence of, or in the presence of, an inert gas. It is preferable that the alkylation uses no inert gas.

In one specific embodiment of the alkylation, 2,5-di(hydroxymethyl)tetrahydrofuran or 2,5-di(hydroxyethyl)tetrahydrofuran is converted to the diether compounds of the general formula (I.2) in the presence of an at least four-fold excess of base in an inert organic solvent and with at least one alkyl bromide R¹—Br and, respectively, R²—Br. In relation to the moieties R¹ and R², reference is made to the previous descriptions. As base, it is preferable to use an alkali metal hydroxide, in particular KOH.

To produce the ester compounds of the general formula (I.3) of the invention, it is preferable to react 2,5-di(hydroxymethyl)tetrahydrofuran or 2,5-di(hydroxyethyl)tetrahydrofuran with at least one acyl halide R¹—(C═O)X and, if R¹ and R² are different, with at least one acyl halide R²—(C═O)X, where X is Br or Cl, in the presence of at least one tertiary amine, to give the compounds of the formula (I.3).

There are also other familiar esterification methods, alongside this process, available to the person skilled in the art, as previously described in relation to the esterification of FDCA and, respectively, 2,5-tetrahydrofurandicarboxylic acid.

The ester compounds of the general formula (I.3) can usually be produced by using any of the tertiary amines familiar to the person skilled in the art. Examples of suitable tertiary amines are:
from the group of the trialkylamines: trimethylamine, triethylamine, tri-n-propylamine, diethylisopropylamine, diisopropylethylamine and the like;
from the group of the N-cycloalkyl-N,N-dialkylamines: dimethylcyclohexylamine and diethylcyclohexylamine;
from the group of the N,N-dialkylanilines: dimethylaniline and diethylaniline;
from the group of the pyridine and quinoline bases: pyridine, α-, β-, and γ-picoline, quinoline and 4-(dimethylamino)pyridine (DMAP).

Preferred tertiary amines are trialkylamines and pyridine bases, in particular triethylamine and 4-(dimethylamino)pyridine (DMAP), and also mixtures thereof.

The esterification can take place at ambient pressure, or at reduced or elevated pressure. It is preferable to carry out the esterification at ambient pressure.

The esterification can be carried out in the absence of, or in the presence of, an organic solvent. It is preferable to carry out the esterification in the presence of an inert organic solvent, as defined previously.

The esterification is usually carried out in the temperature range from 50 to 200° C.

The esterification can take place in the absence of, or in the presence of, an inert gas.

In one preferred embodiment, 2,5-di(hydroxymethyl)tetrahydrofuran is reacted with an acyl chloride R¹—(C═O)Cl in the presence of triethylamine and/or DMAP and of an inert organic solvent to give compounds of the formula (I.3).

The preferred embodiments of the processes of the invention for producing compounds of the general formula (I) use C₇-C₁₂-alkanols as starting materials for the transesterification, esterification, or alkylation, in particular C₈-C₁₁-alkanols.

Preferred C₇-C₁₂-alkanols can be straight-chain or branched compounds, or can be composed of mixtures of straight-chain and branched C₇-C₁₂-alkanols. Among these are n-heptanol, isoheptanol, n-octanol, isooctanol, 2-ethylhexanol, n-nonanol, isononanol, isodecanol, 2-propylheptanol, n-undecanol, isoundecanol, n-dodecanol, and isododecanol. Particularly preferred C₇-C₁₂-alkanols are n-octanol, 2-ethylhexanol, n-nonanol, isononanol, and 2-propylheptanol, in particular isononanol and 2-propylheptanol.

Heptanol

The heptanols needed for the production of the compounds of the invention of the general formula (I) can be straight-chain or branched heptanols or can be composed of mixtures of straight-chain and branched heptanols. It is preferable to use mixtures of branched heptanols, also known as isoheptanol, which are produced via rhodium- or preferably cobalt-catalyzed hydroformylation of propene dimer, obtainable by way of example by the Dimersol® process, and subsequent hydrogenation of the resultant isoheptanals to give an isoheptanol mixture. Because of the process used for its production, the resultant isoheptanol mixture is composed of a plurality of isomers. Substantially straight-chain heptanols can be obtained via rhodium- or preferably cobalt-catalyzed hydroformylation of 1-hexene and subsequent hydrogenation of the resultant n-heptanal to give n-heptanol. The hydroformylation of 1-hexene or of propene dimer can be achieved by methods known per se: compounds used as catalyst in hydroformylation with rhodium catalysts homogeneously dissolved in the reaction medium can be not only uncomplexed rhodium carbonyl compounds which are formed in situ under the conditions of the hydroformylation reaction within the hydroformylation reaction mixture on exposure to synthesis gas, e.g. from rhodium salts, but also complex rhodium carbonyl compounds, in particular complexes with organic phosphines, such as triphenylphosphine, or with organophosphites, preferably chelating biphosphites, as described by way of example in U.S. Pat. No. 5,288,918. Compounds used in the cobalt-catalyzed hydroformylation of these olefins are generally cobalt carbonyl compounds which are homogeneously soluble in the reaction mixture and which are formed in situ from cobalt salts under the conditions of the hydroformylation reaction on exposure to synthesis gas. If the cobalt-catalyzed hydroformylation is carried out in the presence of trialkyl- or triarylphosphines, the desired heptanols are formed directly as hydroformylation product, and there is therefore then no need for further hydrogenation of the aldehyde function.

Examples of suitable processes for the cobalt-catalyzed hydroformylation of 1-hexene or of the hexene isomer mixtures are the established industrial processes explained on pages 162-168 of Falbe, New Syntheses with Carbon Monoxide, Springer, Berlin, 1980, an example being the Ruhrchemie process, the BASF process, the Kuhlmann process, or the Shell process. Whereas the Ruhrchemie, BASF, and Kuhlmann process operate with non-ligand-modified cobalt carbonyl compounds as catalysts and thus give hexanal mixtures, the Shell process (DE-A 1593368) uses, as catalyst, phosphine- or phosphite-ligand-modified cobalt carbonyl compounds which lead directly to the hexanol mixtures because they also have high hydrogenation activity. DEA 2139630, DE-A 2244373, DE-A 2404855, and WO 01014297 provide detailed descriptions of advantageous embodiments for the conduct of the hydroformylation with non-ligand-modified cobalt carbonyl complexes.

The rhodium-catalyzed hydroformylation of 1-hexene or of the hexene isomer mixtures can use the established industrial low-pressure rhodium hydroformylation process with triphenylphosphine-ligand-modified rhodium carbonyl compounds, which is subject matter of U.S. Pat. No. 4,148,830. Non-ligand-modified rhodium carbonyl compounds can serve advantageously as catalyst for the rhodium-catalyzed hydroformylation of long-chain olefins, for example of the hexene isomer mixtures obtained by the processes described above; this differs from the low-pressure process in requiring a higher pressure of from 80 to 400 bar. The conduct of high-pressure rhodium hydroformylation processes of this type is described by way of example in EP-A 695734, EP-B 880494, and EP-B 1047655.

The isoheptanal mixtures obtained after hydroformylation of the hexene isomer mixtures are catalytically hydrogenated in a manner that is per se conventional to give isoheptanol mixtures. For this purpose it is preferable to use heterogeneous catalysts which comprise, as catalytically active component, metals and/or metal oxides of group VI to VIII, or else of transition group I, of the periodic table of the elements, in particular chromium, molybdenum, manganese, rhenium, iron, cobalt, nickel, and/or copper, optionally deposited on a support material, such as Al₂O₃, SiO₂ and/or TiO₂. Catalysts of this type are described by way of example in DE-A 3228881, DE-A 2628987, and DEA 2445303. It is particularly advantageous to carry out the hydrogenation of the isoheptanals with an excess of hydrogen of from 1.5 to 20% above the stoichiometric amount of hydrogen needed for the hydrogenation of the isoheptanals, at temperatures of from 50 to 200° C., and at a hydrogen pressure of from 25 to 350 bar, and for avoidance of side-reactions to add, during the course of the hydrogenation, in accordance with DE-A 2628987, a small amount of water, advantageously in the form of an aqueous solution of an alkali metal hydroxide or alkali metal carbonate, in accordance with the teaching of WO 01087809.

Octanol

For many years, 2-ethylhexanol was the largest-production-quantity plasticizer alcohol, and it can be obtained through the aldol condensation of n-butyraldehyde to give 2-ethylhexanal and subsequent hydrogenation thereof to give 2-ethylhexanol (see Ullmann's Encyclopedia of Industrial Chemistry; 5th edition, vol. A 10, pp. 137-140, VCH Verlagsgesellschaft GmbH, Weinheim 1987).

Substantially straight-chain octanols can be obtained via rhodium- or preferably cobalt-catalyzed hydroformylation of 1-heptene and subsequent hydrogenation of the resultant n-octanal to give n-octanol. The 1-heptene needed for this purpose can be obtained from the Fischer-Tropsch synthesis of hydrocarbons.

By virtue of the production route used for the alcohol isooctanol, it is not a unitary chemical compound, in contrast to 2-ethylhexanol, n-octanol, but instead is an isomer mixture of variously branched C₈-alcohols, for example of 2,3-dimethyl-1-hexanol, 3,5-dimethyl-1-hexanol, 4,5-dimethyl-1-hexanol, 3-methyl-1-heptanol, and 5-methyl-1-heptanol; these can be present in the isooctanol in various quantitative proportions which depend on the production conditions and production processes used. Isooctanol is usually produced via codimerization of propene with butenes, preferably n-butenes, and subsequent hydroformylation of the resultant mixture of heptene isomers. The octanal isomer mixture obtained in the hydroformylation can subsequently be hydrogenated to give the isooctanol in a manner that is conventional per se.

The codimerization of propene with butenes to give isomeric heptenes can advantageously be achieved with the aid of the homogeneously catalyzed Dimersol® process (Chauvin et al; Chem. Ind.; May 1974, pp. 375-378), which uses, as catalyst, a soluble nickel phosphine complex in the presence of an ethylaluminum chlorine compound, for example ethylaluminum dichloride. Examples of phosphine ligands that can be used for the nickel complex catalyst are tributylphosphine, triisopropylphosphine, tricyclohexylphosphine, and/or tribenzylphosphine. The reaction takes place at temperatures of from 0 to 80° C., and it is advantageous here to set a pressure at which the olefins are present in solution in the liquid reaction mixture (Cornils; Hermann: Applied Homogeneous Catalysis with Organometallic Compounds; 2^{nd} edition, vol. 1; pp. 254-259, Wiley-VCH, Weinheim 2002).

In an alternative to the Dimersol® process operated with nickel catalysts homogeneously dissolved in the reaction medium, the codimerization of propene with butenes can also be carried out with a heterogeneous NiO catalyst deposited on a support; heptene isomer distributions obtained here are similar to those obtained in the homogeneously catalyzed process. Catalysts of this type are by way of example used in what is known as the Octol® process (Hydrocarbon Processing, February 1986, pp. 31-33), and a specific heterogeneous nickel catalyst with good suitability for olefin dimerization or olefin codimerization is disclosed by way of example in WO 9514647.

Codimerization of propene with butenes can also use, instead of nickel-based catalysts, heterogeneous Brønsted-acid catalysts; heptenes obtained here are generally more highly branched than in the nickel-catalyzed processes. Examples of catalysts suitable for this purpose are solid phosphoric acid catalysts, e.g. phosphoric-acid-impregnated kieselguhr or diatomaceous earth, these being as utilized in the PolyGas® process for olefin dimerization or olefin oligomerization (Chitnis et al; Hydrocarbon Engineering 10, No. 6—June 2005). Brønsted-acid catalysts that have very good suitability for the codimerization of propene and butenes to give heptenes are zeolites, which are used in the EMOGAS® process, a further development based on the PolyGas® process.

The 1-heptene and the heptene isomer mixtures are converted to n-octanal and, respectively, octanal isomer mixtures by the known processes explained above in connection with the production of n-heptanal and heptanal isomer mixtures, by means of rhodium- or cobalt-catalyzed hydroformylation, preferably cobalt-catalyzed hydroformylation. These are then hydrogenated to give the corresponding octanols, for example by means of a catalyst mentioned above in connection with production of n-heptanol and of isoheptanol.

Nonanol

Substantially straight-chain nonanol can be obtained via rhodium- or preferably cobalt-catalyzed hydroformylation of 1-octene and subsequent hydrogenation of the resultant n-nonanal. The starting olefin 1-octene can be obtained by way of example by way of ethylene oligomerization by means of a nickel complex catalyst that is homogenously soluble in the reaction medium—1,4-butanediol—with, for example, diphenyl-phosphinoacetic acid or 2-diphenylphosphinobenzoic acid as ligand. This process is also known as the Shell Higher Olefins Process or SHOP process (see Weisermel, Arpe: Industrielle Organische Chemie [Industrial organic chemistry]; 5th edition, p. 96; Wiley-VCH, Weinheim 1998).

The alcohol component isononanol needed for the synthesis of the diisononyl esters and diisononyl ethers of the invention is not a unitary chemical compound, but instead is a mixture of variously branched, isomeric C₉-alcohols which can have various degrees of branching, depending on the manner in which they were produced, and also in particular on the starting materials used. The isononanols are generally produced via dimerization of butenes to give isooctene mixtures, subsequent hydroformylation of the isooctene mixtures, and hydrogenation of the resultant isononanal mixtures to give isononanol mixtures, as explained in Ullmann's Encyclopedia of Industrial Chemistry, 5th edition, vol. A1, pp. 291-292, VCH Verlagsgesellschaft GmbH, Weinheim 1995.

Isobutene, cis- and trans-2-butene, and also 1-butene, or a mixture of these butene isomers, can be used as starting material for the production of the isononanols. The dimerization of pure isobutene, mainly catalyzed by means of liquid Brønsted acids, e.g. sulfuric acid or phosphoric acid, or by means of solid Brønsted acids, e.g. phosphoric acid absorbed on kieselguhr, SiO₂, or Al₂O₃, as support material, or zeolites, mainly gives the highly branched compound 2,4,4-trimethylpentene, also termed diisobutylene, which gives highly branched isononanols after hydroformylation and hydrogenation of the aldehyde.

Preference is given to isononanols with a low degree of branching. Isononanol mixtures of this type with little branching are obtained from the linear butenes 1-butene, and cis- and/or trans-2-butene which optionally can also comprise relatively small amounts of isobutene, by way of the route described above involving butene dimerization, hydroformylation of the isooctene, and hydrogenation of the resultant isononanal mixtures. A preferred raw material is what is known as raffinate II, which is obtained from the Ca-cut of a cracker, for example of a steam cracker, after elimination of allenes, acetylenes, and dienes, in particular 1,3-butadiene, via partial hydrogenation thereof to give linear butenes, or removal thereof via extractive distillation, for example by means of N-methylpyrrolidone, and subsequent Brønsted-acid catalyzed removal of the isobutene comprised therein via reaction thereof with methanol or isobutanol by established large-scale-industrial processes with formation of the fuel additive methyl tert-butyl ether (MTBE), or of the isobutyl tert-butyl ether that is used to obtain pure isobutene.

Raffinate II also comprises, alongside 1-butene and cis- and trans-2-butene, n- and isobutane, and residual amounts of up to 5% by weight of isobutene.

The dimerization of the linear butenes or of the butene mixture comprised in raffinate II can be carried out by means of the familiar processes used on a large industrial scale, for example those explained above in connection with the production of isoheptene mixtures, for example by means of heterogeneous, Brønsted-acid catalysts such as those used in the PolyGas® process or EMOGAS® process, by means of the Dimersol® process with use of nickel complex catalysts homogeneously dissolved in the reaction medium, or by means of heterogeneous, nickel(II)-oxide-containing catalysts by the Octol® process or by the process of WO 9514647. The resultant isooctene mixtures are converted to isononanal mixtures by the known processes explained above in connection with the production of heptanal isomer mixtures, by means of rhodium or cobalt-catalyzed hydroformylation, preferably cobalt-catalyzed hydroformylation. These are then hydrogenated to give the suitable isononanol mixtures, for example by means of one of the catalysts mentioned above in connection with the production of isoheptanol.

The resultant isononanol isomer mixtures can be characterized by way of their iso-index, which can be calculated from the degree of branching of the individual, isomeric isononanol components in the isononanol mixture multiplied by the percentage proportion of these in the isononanol mixture: by way of example, n-nonanol contributes the value 0 to the iso-index of an isononanol mixture, methyloctanols (single branching) contribute the value 1, and dimethylheptanols (double branching) contribute the value 2. The higher the linearity, the lower is the iso-index of the relevant isononanol mixture. Accordingly, the iso-index of an isononanol mixture can be determined via gas-chromatographic separation of the isononanol mixture into its individual isomers and attendant quantification of the percentage quantitative proportion of these in the isononanol mixture, determined by standard methods of gas-chromatographic analysis. In order to increase the volatility of the isomeric nonanols and improve the gas-chromatographic separation of these, they are advantageously trimethylsilylated by means of standard methods, for example via reaction with N-methyl-N-trimethylsilyltrifluoracetamide, prior to gas-chromatographic analysis. In order to achieve maximum quality of separation of the individual components during gas-chromatographic analysis, it is preferable to use capillary columns with polydimethylsiloxane as stationary phase. Capillary columns of this type are obtainable commercially, and a little routine experimentation by the person skilled in the art is all that is needed in order to select, from the many different products available commercially, one that has ideal suitability for this separation task.

The inventive diisononyl esters and diisononyl ethers of the general formula (I) have generally been esterified and, respectively, etherified with isononanols with an iso-index of from 0.8 to 2, preferably from 1.0 to 1.8, and particularly preferably from 1.1 to 1.5, and these can be produced by the processes mentioned above.

Merely by way of example, possible compositions of isononanol mixtures of the type that can be used for the production of the inventive compounds diisononyl 2,5-tetrahydrofurandicarboxylate, 2,5-di(hydroxymethyl)tetrahydrofuran diisononanoate, and diisononyl ether of 2,5-di(hydroxymethyl)tetrahydrofuran are stated below, and it should be noted here that the proportions of the isomers individually listed within the isononanol mixture can vary, depending on the composition of the starting material, for example raffinate II, the composition of butenes in which can vary with the production process, and on variations in the production conditions used, for example in the age of the catalysts utilized, and conditions of temperature and of pressure, which require appropriate adjustment.

By way of example, an isononanol mixture produced via cobalt-catalyzed hydroformylation and subsequent hydrogenation from an isooctene mixture produced with use of raffinate II as raw material by means of the catalyst and process in accordance with WO 9514647 can have the following composition:
from 1.73 to 3.73% by weight, preferably from 1.93 to 3.53% by weight, particularly preferably from 2.23 to 3.23% by weight of 3-ethyl-6-methyl-hexanol;
from 0.38 to 1.38% by weight, preferably from 0.48 to 1.28% by weight, particularly preferably from 0.58 to 1.18% by weight of 2,6-dimethylheptanol;
from 2.78 to 4.78% by weight, preferably from 2.98 to 4.58% by weight, particularly preferably from 3.28 to 4.28% by weight of 3,5-dimethylheptanol;
from 6.30 to 16.30% by weight, preferably from 7.30 to 15.30% by weight, particularly preferably from 8.30 to 14.30% by weight of 3,6-dimethylheptanol;
from 5.74 to 11.74% by weight, preferably from 6.24 to 11.24% by weight, particularly preferably from 6.74 to 10.74% by weight of 4,6-dimethylheptanol;
from 1.64 to 3.64% by weight, preferably from 1.84 to 3.44% by weight, particularly preferably from 2.14 to 3.14% by weight of 3,4,5-trimethylhexanol;
from 1.47 to 5.47% by weight, preferably from 1.97 to 4.97% by weight, particularly preferably from 2.47 to 4.47% by weight of 3,4,5-trimethylhexanol, 3-methyl-4-ethylhexanol and 3-ethyl-4-methylhexanol;
from 4.00 to 10.00% by weight, preferably from 4.50 to 9.50% by weight, particularly preferably from 5.00 to 9.00% by weight of 3,4-dimethylheptanol;
from 0.99 to 2.99% by weight, preferably from 1.19 to 2.79% by weight, particularly preferably from 1.49 to 2.49% by weight of 4-ethyl-5-methylhexanol and 3-ethylheptanol;
from 2.45 to 8.45% by weight, preferably from 2.95 to 7.95% by weight, particularly preferably from 3.45 to 7.45% by weight of 4,5-dimethylheptanol and 3-methyloctanol;
from 1.21 to 5.21% by weight, preferably from 1.71 to 4.71% by weight, particularly preferably from 2.21 to 4.21% by weight of 4,5-dimethylheptanol;
from 1.55 to 5.55% by weight, preferably from 2.05 to 5.05% by weight, particularly preferably from 2.55 to 4.55% by weight of 5,6-dimethylheptanol;
from 1.63 to 3.63% by weight, preferably from 1.83 to 3.43% by weight, particularly preferably from 2.13 to 3.13% by weight of 4-methyloctanol;
from 0.98 to 2.98% by weight, preferably from 1.18 to 2.78% by weight, particularly preferably from 1.48 to 2.48% by weight of 5-methyloctanol;
from 0.70 to 2.70% by weight, preferably from 0.90 to 2.50% by weight, particularly preferably from 1.20 to 2.20% by weight of 3,6,6-trimethylhexanol;
from 1.96 to 3.96% by weight, preferably from 2.16 to 3.76% by weight, particularly preferably from 2.46 to 3.46% by weight of 7-methyloctanol;
from 1.24 to 3.24% by weight, preferably from 1.44 to 3.04% by weight, particularly preferably from 1.74 to 2.74% by weight of 6-methyloctanol;
from 0.1 to 3% by weight, preferably from 0.2 to 2% by weight, particularly preferably from 0.3 to 1% by weight of n-nonanol;
from 25 to 35% by weight, preferably from 28 to 33% by weight, particularly preferably from 29 to 32% by weight of other alcohols having 9 and 10 carbon atoms;
with the proviso that the entirety of the components mentioned gives 100% by weight.

In accordance with what has been said above, an isononanol mixture produced via cobalt-catalyzed hydroformylation and subsequent hydrogenation with use of an isooctene mixture produced by means of the PolyGas® process or EMOGAS® process with an ethylene-containing butene mixture as raw material can vary within the range of the compositions below, depending on the composition of the raw material and variations in the reaction conditions used:
from 6.0 to 16.0% by weight, preferably from 7.0 to 15.0% by weight, particularly preferably from 8.0 to 14.0% by weight of n-nonanol;
from 12.8 to 28.8% by weight, preferably from 14.8 to 26.8% by weight, particularly preferably from 15.8 to 25.8% by weight of 6-methyloctanol;
from 12.5 to 28.8% by weight, preferably from 14.5 to 26.5% by weight, particularly preferably from 15.5 to 25.5% by weight of 4-methyloctanol;
from 3.3 to 7.3% by weight, preferably from 3.8 to 6.8% by weight, particularly preferably from 4.3 to 6.3% by weight of 2-methyloctanol;
from 5.7 to 11.7% by weight, preferably from 6.3 to 11.3% by weight, particularly preferably from 6.7 to 10.7% by weight of 3-ethylheptanol;
from 1.9 to 3.9% by weight, preferably from 2.1 to 3.7% by weight, particularly preferably from 2.4 to 3.4% by weight of 2-ethylheptanol;
from 1.7 to 3.7% by weight, preferably from 1.9 to 3.5% by weight, particularly preferably from 2.2 to 3.2% by weight of 2-propylhexanol;
from 3.2 to 9.2% by weight, preferably from 3.7 to 8.7% by weight, particularly preferably from 4.2 to 8.2% by weight of 3,5-dimethylheptanol;
from 6.0 to 16.0% by weight, preferably from 7.0 to 15.0% by weight, particularly preferably from 8.0 to 14.0% by weight of 2,5-dimethylheptanol;
from 1.8 to 3.8% by weight, preferably from 2.0 to 3.6% by weight, particularly preferably from 2.3 to 3.3% by weight of 2,3-dimethylheptanol;
from 0.6 to 2.6% by weight, preferably from 0.8 to 2.4% by weight, particularly preferably from 1.1 to 2.1% by weight of 3-ethyl-4-methylhexanol;
from 2.0 to 4.0% by weight, preferably from 2.2 to 3.8% by weight, particularly preferably from 2.5 to 3.5% by weight of 2-ethyl-4-methylhexanol;
from 0.5 to 6.5% by weight, preferably from 1.5 to 6% by weight, particularly preferably from 1.5 to 5.5% by weight of other alcohols having 9 carbon atoms;
with the proviso that the entirety of the components mentioned gives 100% by weight.

Decanol

The alcohol component isodecanol needed for the production of the compounds of the general formula (I) of the invention is not a unitary chemical compound, but instead is a complex mixture of variously branched, isomeric decanols.

These are generally produced via nickel- or Brønsted-acid-catalyzed trimerization of propylene, for example by the PolyGas® process or the EMOGAS® process explained above, subsequent hydroformylation of the resultant isononene isomer mixture by means of homogeneous rhodium or cobalt carbonyl catalysts, preferably by means of cobalt carbonyl catalysts, and hydrogenation of the resultant isodecanal isomer mixture, e.g. by means of the catalysts and processes mentioned above in connection with the production of C₇-C₉-alcohols (Ullmann's Encyclopedia of Industrial Chemistry; 5th edition, vol. A1, p. 293, VCH Verlagsgesellschaft GmbH, Weinheim 1985). The resultant isodecanol generally has a high degree of branching.

The 2-propylheptanol needed for the production of the di(2-propylheptyl) esters or di(2-propylheptyl) ethers of the invention can be pure 2-propylheptanol or can be a propylheptanol isomer mixture of the type generally formed during the industrial production of 2-propylheptanol and generally also called 2-propylheptanol.

Pure 2-propylheptanol can be obtained via aldol condensation of n-valeraldehyde and subsequent hydrogenation of the resultant 2-propylheptanal, for example in accordance with U.S. Pat. No. 2,921,089. By virtue of the production process, commercially obtainable 2-propylheptanol generally comprises, alongside the main component 2-propylheptanol, one or more of the following isomers of 2-propylheptanol: 2-propyl-4-methylhexanol, 2-propyl-5-methylhexanol, 2-isopropylheptanol, 2-isopropyl-4-methyl-hexanol, 2-isopropyl-5-methylhexanol, and/or 2-propyl-4,4-dimethylpentanol. The presence of other isomers of 2-propylheptanol, for example 2-ethyl-2,4-dimethylhexanol, 2-ethyl-2-methylheptanol, and/or 2-ethyl-2,5-dimethylhexanol, in the 2-propylheptanol is possible; but because the rates of formation of the aldehydic precursors of these isomers in the aldol condensation are low, the amounts of these present in the 2-propylheptanol are only trace amounts, if they are present at all, and they play practically no part in determining the plasticizer properties of the compounds produced from these 2-propylheptanol isomer mixtures.

Various hydrocarbon sources can be utilized as starting material for the production of 2-propylheptanol, for example 1-butene, 2-butene, raffinate I—an alkane/alkene mixture which is obtained from the C₄-cut of a cracker after removal of allenes, of acetylenes, and of dienes and which also comprises, alongside 1- and 2-butene, considerable amounts of isobutene—or raffinate II, which is obtained from raffinate I via removal of isobutene and then comprises, as olefin components other than 1- and 2-butene, only small proportions of isobutene. It is also possible, of course, to use mixtures of raffinate I and raffinate II as raw material for the production of 2-propylheptanol. These olefins or olefin mixtures can be hydroformylated by methods that are per se conventional with cobalt or rhodium catalysts, and 1-butene here gives a mixture of n- and isovaleraldehyde—the term isovaleraldehyde designating the compound 2-methylbutanal, the n/iso ratio of which can vary within relatively wide limits, depending on catalyst used and on hydroformylation conditions. By way of example, when a triphenylphosphine-modified homogeneous rhodium catalyst (Rh/TPP) is used, n- and isovaleraldehyde are formed in an n/iso ratio that is generally from 10:1 to 20:1 from 1-butene, whereas when rhodium hydroformylation catalysts modified with phosphite ligands are used, for example in accordance with U.S. Pat. No. 5,288,918 or WO 05028407, or when rhodium hydroformylation catalysts modified with phosphoamidite ligands are used, for example in accordance with WO 02083695, n-valeraldehyde is formed almost exclusively. While the Rh/TPP catalyst system converts 2-butene only very slowly in the hydroformylation, and most of the 2-butene can therefore be reclaimed from the hydroformylation mixture, 2-butene is successfully hydroformylated with the phosphite-ligand- or phosphorus amidite ligand-modified rhodium catalysts mentioned, the main product formed being n-valeraldehyde. In contrast, isobutene comprised within the olefinic raw material is hydroformylated at varying rates by practically all catalyst systems to 3-methylbutanal and, in the case of some catalysts, to a lesser extent to pivalaldehyde.

The C₅-aldehydes obtained in accordance with starting materials and catalysts used, i.e. n-valeraldehyde optionally mixed with isovaleraldehyde, 3-methylbutanal, and/or pivalaldehyde, can be separated, if desired, completely or to some extent by distillation into the individual components prior to the aldol condensation, and here again there is therefore a possibility of influencing and of controlling the composition of isomers of the C₁₀-alcohol component of the ester mixtures and ether mixtures of the invention. Equally, it is possible that the C₅-aldehyde mixture formed during the hydroformylation is introduced into the aldol condensation without prior isolation of individual isomers. If n-valeraldehyde is used in the aldol condensation, which can be carried out by means of a basic catalyst, for example an aqueous solution of sodium hydroxide or of potassium hydroxide, for example by the processes described in EP-A 366089, U.S. Pat. No. 4,426,524, or U.S. Pat. No. 5,434,313, 2-propylheptanal is produced as sole condensate, whereas if a mixture of isomeric C₅-aldehydes is used the product comprises an isomer mixture of the products of the homoaldol condensation of identical aldehyde molecules and of the crossed aldol condensation of different valeraldehyde isomers. The aldol condensation can, of course, be controlled via targeted reaction of individual isomers in such a way that a single aldol condensation isomer is formed predominantly or entirely. The relevant aldol condensates can then be hydrogenated with conventional hydrogenation catalysts, for example those mentioned above for the hydrogenation of aldehydes, to give the corresponding alcohols or alcohol mixtures, usually after preferably distillative isolation from the reaction mixture and, if desired, distillative purification.

As mentioned above, the compounds di(propylheptyl) 2,5-tetrahydrofurandicarboxylate, 2,5-di(hydroxymethyl)tetrahydrofuran di(2-propyl)heptanoate, and the di-(2-propyl)heptyl ether of 2,5-di(hydroxymethyl)tetrahydrofuran can have been esterified and, respectively, etherified with pure 2-propylheptanol. However, the production of these esters or ethers generally uses mixtures of 2-propylheptanol with the propylheptanol isomers mentioned in which the content of 2-propylheptanol is at least 50% by weight, preferably from 60 to 98% by weight, and particularly preferably from 80 to 95% by weight, in particular from 85 to 95% by weight.

Suitable mixtures of 2-propylheptanol with the propylheptanol isomers comprise by way of example those of from 60 to 98% by weight of 2-propylheptanol, from 1 to 15% by weight of 2-propyl-4-methylhexanol, and from 0.01 to 20% by weight of 2-propyl-5-methylhexanol, and from 0.01 to 24% by weight of 2-isopropylheptanol, where the sum of the proportions of the individual constituents does not exceed 100% by weight. It is preferable that the proportions of the individual constituents give a total of 100% by weight.

Other suitable mixtures of 2-propylheptanol with the propylheptanol isomers comprise by way of example those of from 75 to 95% by weight of 2-propylheptanol, from 2 to 15% by weight of 2-propyl-4-methylhexanol, from 1 to 20% by weight of 2-propyl-5-methylhexanol, from 0.1 to 4% by weight of 2-isopropylheptanol, from 0.1 to 2% by weight of 2-isopropyl-4-methylhexanol, and from 0.1 to 2% by weight of 2-isopropyl-5-methylhexanol, where the sum of the proportions of the individual constituents does not exceed 100% by weight. It is preferable that the proportions of the individual constituents give a total of 100% by weight.

Preferred mixtures of 2-propylheptanol with the propylheptanol isomers comprise those with from 85 to 95% by weight of 2-propylheptanol, from 5 to 12% by weight of 2-propyl-4-methylhexanol, and from 0.1 to 2% by weight of 2-propyl-5-methylhexanol, and from 0.01 to 1% by weight of 2-isopropylheptanol, where the sum of the proportions of the individual constituents does not exceed 100% by weight. It is preferable that the proportions of the individual constituents give a total of 100% by weight.

When the 2-propylheptanol isomer mixtures are used instead of pure 2-propylheptanol for the production of the di(2-propylheptyl) esters or di(2-propylheptyl) ethers of the invention, the isomer composition of the alkyl ester groups and, respectively, alkyl ether groups is practically the same as the composition of the propylheptanol isomer mixtures used for the esterification.

Undecanol

The undecanols needed for the production of the compounds of the general formula (I) of the invention can be straight-chain or branched or can be composed of a mixture of straight-chain and branched undecanols. It is preferable to use, as alcohol component of the diundecyl esters or diundecyl ethers of the invention, mixtures of branched undecanols, which are also termed isoundecanol.

Substantially straight-chain undecanol can be obtained via rhodium- or preferably cobalt-catalyzed hydroformylation of 1-decene and subsequent hydrogenation of the resultant n-undecanal. The starting olefin 1-decene is produced by way of the SHOP process mentioned previously for the production of 1-octene.

For the production of branched isoundecanol, the 1-decene obtained in the SHOP process can be subjected to skeletal isomerization, for example by means of acidic zeolitic molecular sieves, as described in WO 9823566, whereupon mixtures of isomeric decenes are formed, rhodium- or preferably cobalt-catalyzed hydroformylation of which, with subsequent hydrogenation of the resultant isoundecanal mixtures, gives the isoundecanol used for the production of the compounds of the invention. Hydroformylation of 1-decene or of isodecene mixtures by means of rhodium or cobalt catalysis can be achieved as described previously in connection with the synthesis of C₇-C₁₀-alcohols. Similar considerations apply to the hydrogenation of n-undecanal or of isoundecanal mixtures to give n-undecanol and, respectively, isoundecanol.

After distillative purification of the hydrogenation product, the resultant C₇-C₁₁-alkyl alcohols or a mixture of these can be used as described above for the production of the diester derivatives or diether derivatives of the general formula (I).

Dodecanol

Substantially straight-chain dodecanol can be obtained advantageously by way of the Alfol® process or Epal® process. These processes include the oxidation and hydrolysis of straight-chain trialkylaluminum compounds which are constructed stepwise by way of a plurality of ethylation reactions, starting from triethylaluminum, with use of Ziegler-Natta catalysts. The desired n-dodecanol can be obtained from the resultant mixtures of substantially straight-chain alkyl alcohols of varying chain length after distillative discharge of the C₁₂-alkyl alcohol fraction.

Alternatively, n-dodecanol can also be produced via hydrogenation of natural fatty acid methyl esters, for example from coconut oil.

Branched isododecanol can be obtained by analogy with the processes described previously for the codimerization and/or oligomerization of olefins with subsequent hydroformylation and hydrogenation of the isoundecene mixtures. After distillative purification of the hydrogenation product, the resultant isododecanols or mixtures of these can be used as described above for the production of the diester derivatives or diether derivatives of the general formula (I).

The furan-2,5-dicarboxylic acid (FDCA, CAS No. 3238-40-2) needed as starting material for the preferred processes for producing compounds of the general formula (I) can either be purchased commercially or can be produced by synthesis routes known from the literature: possibilities for synthesis are found in the publication by Lewkowski et al. published on the Internet with the title “Synthesis, Chemistry and Application of 5-hydroxymethylfurfural and its derivatives” (Lewkowski et al., ARKIVOC 2001 (i), pp. 17-54, ISSN 1424-6376). A feature common to most of these syntheses is acid-catalyzed reaction of carbohydrates, particularly glucose and fructose, preferably fructose, to give 5-hydroxymethylfurfural (5-HMF), which can be separated from the reaction mixture by using technical processes such as a two-phase method. Appropriate results have been described by way of example by Leshkov et al. in Science 2006, vol. 312, pp. 1933-1937, and by Zhang et al, in Angewandte Chemie 2008, vol. 120, pp. 9485-9488. 5-HMF can then be oxidized to FDCA in a further step, as cited by way of example by Christensen in ChemSusChem 2007, vol. 1, pp. 75-78.

2,5-Bis(hydroxymethyl)tetrahydrofuran (CAS No. 104-80-3) can likewise either be purchased or can be synthesized. The syntheses described start from 5-HMF, which can be reduced in two steps by way of 2,5-bis(hydroxymethyl)furan (2,5-BHF) or directly to give 2,5-di(hydroxymethyl)tetrahydrofuran (Lewkowski et al., ARKIVOC 2001 (i), pp. 17-54, ISSN 1424-6376).

2,5-Bis(hydroxyethyl)tetrahydrofuran can be obtained via reduction of methyl 2,5-furandiacetate. Methyl 2,5-furandiacetate can be synthesized by way of suitable reactions familiar to the person skilled in the art from 2,5-bis(hydroxymethyl)furan (2,5-BHF), for example by analogy with the process described by Rau et al. in Liebigs Ann. Chem., vol. 1984 (8. 1984), pp. 1504-1512, ISSN 0947-3440. Here, 2,5-bis(chloromethyl)furan is prepared from 2-5-BHF via reaction with thionyl chloride, and is reacted via exposure to KCN in benzene in the presence of [18]-crown-6 to give 2,5-bis(cyanomethyl)furan. 2,5-bis(cyanomethyl)furan can then be hydrolyzed to give 2,5-furandiacetic acid and esterified with methanol to give the dimethyl ester, or can be converted directly to methyl 2,5-furandiacetate via alcoholysis with methanol (pinner reaction). Methyl 2,5-furandiacetate can then either be first hydrogenated to dimethyl tetrahydro-2,5-furandiacetate (by analogy with steps b2) and, respectively, c1)) or can be reduced directly to 2,5-bis(hydroxyethyl)tetrahydrofuran.

Methyl 2,5-furandiacetate can likewise be prepared by analogy with the process described by Kern et al. in Liebigs Ann. Chem., vol. 1985 (6. 1985), pp. 1168-1174, ISSN 0947-3440.

Plasticizer Composition

The compounds of the general formula (I) of the invention feature very good compatibility with a wide variety of plasticizers. They are specifically suitable in combination with other plasticizers which have gelling properties that still require improvement, in order to improve gelling performance: they permit reduction of the temperature required for the gelling of a thermoplastic polymer, and/or can increase the gelling rate of plasticizer compositions.

If there are specific or complex requirements necessary for an application, for example high low-temperature resilience, high resistance to extraction or to migration, or very low plasticizer volatility, it can be advantageous to use plasticizer compositions for plasticizing thermoplastic polymers. This is true in particular for flexible-PVC applications.

The invention therefore also provides plasticizer compositions which comprise at least one compound of the general formula (I) and at least one plasticizer different from the compounds (I).

In relation to suitable and preferred compounds of the general formula (I) for producing plasticizer compositions, reference is made to the entirety of the suitable and preferred compounds of the general formula (I) described previously. It is preferable that the plasticizer compositions of the invention comprise at least one compound of the general formula (I) in which R¹ and R² are mutually independently unbranched or branched C₇-C₁₂-alkyl, in particular isononyl, 2-propylheptyl, or 2-ethylhexyl. A compound of the general formula (I) specifically suitable for producing plasticizer compositions is di(2-propylheptyl) 2,5-tetrahydrofurandicarboxylate.

It is preferable that the additional plasticizer different from the compounds of the general formula (I) is one selected from dialkyl phthalates, alkyl aralkyl phthalates, dialkyl terephthalates, trialkyl trimellitates, dialkyl adipates, alkyl benzoates, dibenzoic esters of glycols, hydroxybenzoic esters, esters of saturated mono- and dicarboxylic acids, esters of unsaturated dicarboxylic acids, amides and esters of aromatic sulfonic acids, alkylsulfonic esters, glycerol esters, isosorbide esters, phosphoric esters, citric triesters, alkylpyrrolidone derivatives, 2,5-furandicarboxylic esters, 2,5-tetrahydrofurandicarboxylic esters different from compounds (I), epoxidized vegetable oils based on triglycerides and saturated or unsaturated fatty acids, polyesters derived from aliphatic and aromatic polycarboxylic acids with polyhydric alcohols.

Preferred dialkyl phthalates have mutually independently from 4 to 13 carbon atoms, preferably from 8 to 13 carbon atoms, in the alkyl chains. An example of a preferred alkyl aralkyl phthalate is benzyl butyl phthalate. It is preferable that the dialkyl terephthalates have mutually independently in each case from 4 to 13 carbon atoms, in particular from 7 to 11 carbon atoms, in the alkyl chains. Examples of preferred dialkyl terephthalates are dialkyl di(n-butyl)terephthalates, dialkyl di(2-ethylhexyl) terephthalates, dialkyl di(isononyl)terephthalates and dialkyl di(2-propylheptyl) terephthalates. It is preferable that the trialkyl trimellitates have mutually independently in each case from 4 to 13 carbon atoms, in particular from 7 to 11 carbon atoms, in the alkyl chains. It is preferable that the esters of saturated mono- and dicarboxylic acids are esters of acetic acid, butyric acid, valeric acid, succinic acid, adipic acid, sebacic acid, lactic acid, malic acid, or tartaric acid. It is preferable that the dialkyl adipates have mutually independently in each case from 4 to 13 carbon atoms, in particular from 6 to 10 carbon atoms, in the alkyl chains. It is preferable that the esters of unsaturated dicarboxylic acids are esters of maleic acid and of fumaric acid. It is preferable that the alkyl benzoates have mutually independently in each case from 7 to 13 carbon atoms, in particular from 9 to 13 carbon atoms, in the alkyl chains. Examples of preferred alkyl benzoates are isononyl benzoate, isodecyl benzoate and 2-propylheptyl benzoate. Preferred dibenzoic esters of glycols are diethylene glycol dibenzoate and dibutylene glycol dibenzoate. Preferred alkylsulfonic esters preferably have an alkyl moiety having from 8 to 22 carbon atoms. Among these are by way of example the phenyl and cresyl esters of pentadecylsulfonic acid. Preferred isosorbide esters are isosorbide diesters, preferably esterified mutually independently in each case with C₅-C₁₃-carboxylic acids. Preferred phosphoric esters are tri-2-ethylhexyl phosphate, trioctyl phosphate, triphenyl phosphate, isodecyl diphenyl phosphate, 2-ethylhexyl diphenyl phosphate, and bis(2-ethylhexyl) phenyl phosphate. The OH group in the citric triesters can be present in free or carboxylated form, preferably in acetylated form. It is preferable that the alkyl moieties of the citric triesters have mutually independently from 4 to 8 carbon atoms, in particular from 6 to 8 carbon atoms. Preference is given to alkylpyrrolidone derivatives having alkyl moieties of from 4 to 18 carbon atoms. Preferred dialkyl 2,5-furandicarboxylates have mutually independently in each case from 4 to 13 carbon atoms, preferably from 8 to 13 carbon atoms, in the alkyl chains. The epoxidized vegetable oils are by way of example preferably epoxidized fatty acids from epoxidized soybean oil, obtainable with trademarks reFlex™ from PolyOne, USA, Proviplast™ PLS Green 5 and Proviplast™ PLS Green 8 from Proviron, Belgium, and Drapex Alpha™ from Galata, USA. It is preferable that the polyesters derived from aliphatic and aromatic polycarboxylic acids are polyesters of adipic acid with polyhydric alcohols, in particular dialkylene glycol polyadipates having from 2 to 6 carbon atoms in the alkylene moiety.

In all of the abovementioned cases, the alkyl moieties can in each case be linear or branched and in each case identical or different. Reference is made to the general descriptions relating to suitable and preferred alkyl moieties in the introduction.

In one particularly preferred embodiment, the plasticizer compositions of the invention comprise at least one plasticizer different from the compounds (I) and selected from dialkyl adipates having from 4 to 9 carbon atoms in the side chain.

In another particularly preferred embodiment, the plasticizer compositions of the invention comprise at least one C₅-C₁₁-dialkyl ester of 2,5-furandicarboxylic acid. Particular preference is given to the C₇-C₁₀-dialkyl esters of 2,5-furandicarboxylic acid.

Suitable and preferred dialkyl esters of 2,5-furandicarboxylic acid are described in WO 2012/113608 (C₅-dialkyl esters), WO 2012/113609 (C₇-dialkyl esters), WO 2011/023490 (C₉-dialkyl esters), and WO 2011/023491 (C₁₀-dialkyl esters). The dihexyl, di(2-ethylhexyl), and di(2-octyl) esters of 2,5-furandicarboxylic acid and their production are described by R. D. Sanderson et al. in J. Appl. Pol. Sci., 1994, vol. 53, 1785-1793. The entire disclosure of those documents is incorporated here by way of reference.

Particularly preferred dialkyl esters of 2,5-furandicarboxylic acid are the isomeric nonyl esters of 2,5-furandicarboxylic acid disclosed in WO 2011/023490. The isomeric nonyl moieties here preferably derive from a mixture of isomeric nonanols as described in WO 2011/023490, page 6, line 32 to page 10, line 15.

In another particularly preferred embodiment, the plasticizer compositions of the invention comprise at least one plasticizer different from the compounds (I) and preferably selected from C₄-C₅-dialkyl esters of 2,5-tetrahydrofurandicarboxylic acid and the C₄-C₅-dialkyl ester derivatives of 2,5-di(hydroxymethyl)tetrahydrofuran or of 2,5-di(hydroxyethyl)tetrahydrofuran. Particular preference is given to the C₄-C₅-dialkyl esters of 2,5-tetrahydrofurandicarboxylic acid, in particular di(isobutyl) 2,5-tetrahydrofurandicarboxylate and di(n-butyl) 2,5-tetrahydrofurandicarboxylate.

Molding Compositions

The present invention further provides a molding composition comprising at least one thermoplastic polymer and at least one compound of the general formula (I).

Thermoplastic polymers that can be used are any of the thermoplastically processable polymers. In particular, these thermoplastic polymers are those selected from:
homo- and copolymers which comprise at least one copolymerized monomer selected from C₂-C₁₀-monoolefins, such as ethylene or propylene, 1,3-butadiene, 2-chloro-1,3-butadiene, vinyl alcohol and its C₂-C₁₀-alkyl esters, vinyl chloride, vinylidene chloride, vinylidene fluoride, tetrafluoroethylene, glycidyl acrylate, glycidyl methacrylate, acrylates and methacrylates with alcohol components of branched and unbranched C₁-C₁₀-alcohols, vinylaromatics, such as polystyrene, (meth)acrylonitrile, α,β-ethylenically unsaturated mono- and dicarboxylic acids, and maleic anhydride;
homo- and copolymers of vinyl acetals;
polyvinyl esters;
polycarbonates (PCs);
polyesters, such as polyalkylene terephthalates, polyhydroxyalkanoates (PHAs), polybutylene succinates (PBSs), polybutylene succinate adipates (PBSAs);
polyethers;
polyether ketones;
thermoplastic polyurethanes (TPUs);
polysulfides;
polysulfones;
   and mixtures thereof.

Mention may be made by way of example of polyacrylates having identical or different alcohol moieties from the group of the C₄-C₈-alcohols, particularly of butanol, hexanol, octanol, and 2-ethylhexanol, polymethyl methacrylate (PMMA), methyl methacrylate-butyl acrylate copolymers, acrylonitrile-butadiene-styrene copolymers (ABSs), ethylene-propylene copolymers, ethylene-propylene-diene copolymers (EPDMs), polystyrene (PS), styrene-acrylonitrile copolymers (SANs), acrylonitrile-styrene-acrylate (ASA), styrene-butadiene-methyl methacrylate copolymers (SBMMAs), styrene-maleic anhydride copolymers, styrene-methacrylic acid copolymers (SMAs), polyoxymethylene (POM), polyvinyl alcohol (PVAL), polyvinyl acetate (PVA), polyvinyl butyral (PVB), polycaprolactone (PCL), polyhydroxybutyric acid (PHB), polyhydroxyvaleric acid (PHV), polylactic acid (PLA), ethylcellulose (EC), cellulose acetate (CA), cellulose propionate (CP), and cellulose acetate/butyrate (CAB).

It is preferable that the at least one thermoplastic polymer comprised in the molding composition of the invention is polyvinyl chloride (PVC), polyvinyl butyral (PVB), homo- and copolymers of vinyl acetate, homo- and copolymers of styrene, polyacrylates, thermoplastic polyurethanes (TPUs), or polysulfides.

The present invention further provides molding compositions comprising at least one elastomer and at least one compound of the general formula (I).

It is preferable that the elastomer comprised in the molding compositions of the invention is at least one natural rubber (NR), at least one rubber produced by a synthetic route, or a mixture thereof. Examples of preferred rubbers produced by a synthetic route are polyisoprene rubber (IR), styrene-butadiene rubber (SBR), butadiene rubber (BR), nitrile-butadiene rubber (NBR), and chloroprene rubber (CR).

Preference is given to rubbers or rubber mixtures which can be vulcanized with sulfur.

For the purposes of the invention, the content (% by weight) of elastomer in the molding compositions is from 20 to 99%, preferably from 45 to 95%, particularly preferably from 50 to 90%, and in particular from 55 to 85%.

The molding composition of the invention can comprise, alongside at least one elastomer and at least one tetrahydrofuran derivative of the general formula (I), at least one plasticizer different from the compounds (I).

Suitable plasticizers different from the compounds (I) are those of the type already defined above.

For the purposes of the invention, the molding compositions which comprise at least one elastomer can comprise other suitable additives, in addition to the above constituents. By way of example, the materials may comprise reinforcing fillers, such as carbon black or silicon dioxide, other fillers, a methylene donor, such as hexamethylenetetraamine (HMT), a methylene acceptor, such as phenolic resins modified with Cardanol (from cashew nuts), a vulcanizing agent or crosslinking agent, a vulcanizing accelerator or crosslinking accelerator, activators, various types of oil, antioxidants, and other various additives which by way of example can be mixed into tire compositions and into other rubber compositions.

Specifically, the at least one thermoplastic polymer comprised in the molding composition of the invention is polyvinyl chloride (PVC).

Polyvinyl chloride is obtained via homopolymerization of vinyl chloride. The polyvinyl chloride (PVC) used in the invention can by way of example be produced via suspension polymerization, microsuspension polymerization, emulsion polymerization, or bulk polymerization. The production of PVC via polymerization of vinyl chloride, and also the production and composition of plasticized PVC, are described by way of example in “Becker/Braun, Kunststoff-Handbuch” [Plastics Handbook], vol. 2/1: Polyvinylchlorid [Polyvinyl chloride], 2nd edn., Carl Hanser Verlag, Munich.

The K value, which characterizes the molar mass of the PVC, and is determined in accordance with DIN 53726, is mostly from 57 to 90 for the PVC plasticized in the invention, preferably from 61 to 85, in particular from 64 to 75.

For the purposes of the invention, the content of PVC in the mixtures is from 20 to 99% by weight, preferably from 45 to 95% by weight, particularly preferably from 50 to 90% by weight, and in particular from 55 to 85% by weight.

At least one plasticizer different from the compounds (I) can be comprised in the molding composition of the invention, alongside at least one thermoplastic polymer and at least one tetrahydrofuran derivative of the general formula (I).

The content of the at least one plasticizer different from the compounds (I) in the molding composition of the invention is from 10 to 90% by weight, preferably from 20 to 85% by weight, and particularly preferably from 50 to 80% by weight, based on the total amount of plasticizer comprised in the molding composition.

Suitable plasticizers different from the compounds (I) are those of the type already defined above.

It is particularly preferable that the at least one additional plasticizer comprised in the molding composition of the invention is selected from dialkyl adipates having from 4 to 9 carbon atoms in the side chain and 2,5-furandicarboxylic esters having from 4 to 10 carbon atoms in the side chain, where the ester groups can have either the same or a different number of carbon atoms.

Amounts of plasticizer used differ in accordance with the choice of thermoplastic polymer or thermoplastic polymer mixture comprised in the molding composition. The total plasticizer content in the molding composition is generally from 0.5 to 300 phr (parts per 100 resin=parts by weight per 100 parts by weight of polymer), preferably from 0.5 to 130 phr, particularly preferably from 1 to 35 phr.

If the thermoplastic polymer in the molding compositions of the invention is polyvinyl chloride and if plasticizer used is exclusively at least one of the (C₇-C₁₂)-dialkyl esters of tetrahydrofurandicarboxylic acid of the invention, total plasticizer content in the molding composition is from 5 to 300 phr, preferably from 10 to 100 phr, and particularly preferably from 30 to 70 phr.

If the thermoplastic polymer in the molding compositions of the invention is polyvinyl chloride and if plasticizer mixtures comprising at least one compound of the general formula (I) and comprising at least one plasticizer different from the compounds (I) are used, the total plasticizer content in the molding composition is from 1 to 400 phr, preferably from 5 to 130 phr, particularly preferably from 10 to 100 phr, and in particular from 15 to 85 phr.

If the polymer in the molding compositions of the invention is rubbers, the total plasticizer content in the molding composition is from 1 to 60 phr, preferably from 1 to 40 phr, particularly preferably from 2 to 30 phr.

Molding Composition Additives

For the purposes of the invention, the molding compositions comprising at least one thermoplastic polymer can comprise other suitable additives. By way of example, the materials can comprise stabilizers, lubricants, fillers, pigments, flame retardants, light stabilizers, blowing agents, polymeric processing aids, impact modifiers, optical brighteners, antistatic agents, or biostabilizers.

Some suitable additives are described in more detail below. However, the examples listed do not represent any restriction of the molding compositions of the invention, but instead serve merely for illustration. All data relating to content are in % by weight, based on the entire molding composition.

Stabilizers that can be used are any of the conventional PVC stabilizers in solid and liquid form, for example conventional Ca/Zn, Ba/Zn, Pb, or Sn stabilizers, and also acid-binding phyllosilicates, such as hydrotalcite.

The molding compositions of the invention can have from 0.05 to 7% content of stabilizers, preferably from 0.1 to 5%, particularly preferably from 0.2 to 4%, and in particular from 0.5 to 3%.

Lubricants are intended to be effective between the PVC pastilles, and to counteract frictional forces during mixing, plastification, and deformation.

The molding compositions of the invention can comprise, as lubricants, any of the lubricants conventionally used for the processing of plastics. Examples of those that can be used are hydrocarbons, such as oils, paraffins, and PE waxes, fatty alcohols having from 6 to 20 carbon atoms, ketones, carboxylic acids, such as fatty acids and montanic acid, oxidized PE wax, metal salts of carboxylic acids, carboxamides, and also carboxylic esters, for example with the following alcohols: ethanol, fatty alcohols, glycerol, ethanediol, and pentaerythritol, and with long-chain carboxylic acids as acid component.

The molding compositions of the invention can have from 0.01 to 10% lubricant content, preferably from 0.05 to 5%, particularly preferably from 0.1 to 3%, and in particular from 0.2 to 2%.

Fillers have an advantageous effect primarily on the compressive strength, tensile strength, and flexural strength, and also the hardness and heat resistance, of plasticized PVC.

For the purposes of the invention, the molding compositions can also comprise fillers such as carbon black and other organic fillers such as natural calcium carbonates, for example chalk, limestone, and marble, dolomite, silicates, silica, sand, diatomaceous earth, aluminum silicates, such as kaolin, mica, and feldspat, and synthetic calcium carbonates. It is preferable to use the following as fillers: calcium carbonates, chalk, dolomite, kaolin, silicates, talc powder, or carbon black.

The molding compositions of the invention can have from 0.01 to 80% content of fillers, preferably from 0.1 to 60%, particularly preferably from 0.5 to 50%, and in particular from 1 to 40%.

The molding compositions of the invention can also comprise pigments in order to adapt the resultant product to be appropriate to various possible uses.

For the purposes of the present invention, it is possible to use either inorganic pigments or organic pigments. Examples of inorganic pigments that can be used are cadmium pigments, such as CdS, cobalt pigments, such as CoO/Al₂O₃, and chromium pigments, such as Cr₂O₃. Examples of organic pigments that can be used are monoazo pigments, condensed azo pigments, azomethine pigments, anthraquinone pigments, quinacridones, phthalocyanine pigments, dioxazine pigments, and aniline pigments.

The molding compositions of the invention can have from 0.01 to 10% content of pigments, preferably from 0.05 to 5%, particularly preferably from 0.1 to 3%, and in particular from 0.5 to 2%.

In order to reduce flammability and to reduce smoke generation during combustion, the molding compositions of the invention can also comprise flame retardants.

Examples of flame retardants that can be used are antimony trioxide, phosphate esters, chloroparaffin, aluminum hydroxide, boron compounds, molybdenum trioxide, ferrocene, calcium carbonate, and magnesium carbonate.

The molding compositions of the invention can have from 0.01 to 10% content of flame retardants, preferably from 0.1 to 8%, particularly preferably from 0.2 to 5%, and in particular from 0.5 to 2%.

The molding compositions can also comprise light stabilizers in order to protect items produced from the molding compositions of the invention from surface damage due to the effect of light.

For the purposes of the present invention it is possible by way of example to use hydroxybenzophenones or hydroxyphenylbenzotriazoles.

The molding compositions of the invention can have from 0.01 to 7% content of light stabilizers, preferably from 0.1 to 5%, particularly preferably from 0.2 to 4%, and in particular from 0.5 to 3%.

Plastisol Applications

As described already, the good gelling properties of the compounds of the invention make them advantageous for producing plastisols.

Plastisols can be produced from various plastics. In one preferred embodiment, the plastisols of the invention are a PVC plastisol.

The plastisols of the invention can optionally comprise, alongside at least one plastic and at least one tetrahydrofuran derivative of the general formula (I), at least one plasticizer different from the compounds (I).

The proportion of the additional at least one plasticizer different from the compounds (I) in the plastisol is from 10 to 90% by weight, preferably from 20 to 85% by weight, and particularly preferably from 50 to 80% by weight, based on the total amount of plasticizer comprised in the plastisol.

In the case of PVC plastisols which comprise, as plasticizer, exclusively at least one of the (C₇-C₁₂)-dialkyl esters of tetrahydrofurandicarboxylic acid of the invention, the total plasticizer content is usually from 5 to 300 phr, preferably from 10 to 100 phr.

In the case of PVC plastisols which comprise, as plasticizer, at least one compound of the general formula (I) and at least one plasticizer different from the compounds (I), the total plasticizer content is usually from 5 to 400 phr, preferably from 50 to 200 phr. Plastisols are usually converted to the form of the finished product at ambient temperature via various processes, such as spreading processes, casting processes, such as the slush molding process or rotomolding process, dip-coating process, spray process, and the like. Gelling then takes place via heating, whereupon cooling gives a homogeneous product with relatively high or relatively low flexibility.

PVC plastisols are particularly suitable for producing PVC foils, for producing seamless hollow bodies, for producing gloves, and for use in the textile sector, e.g. for textile coatings.

Molding Composition Applications

The molding composition of the invention is preferably used for producing moldings and foils. Among these are in particular tooling; apparatuses; piping; cables; hoses, for example plastic hoses, water hoses, and irrigation hoses, industrial rubber hoses, or chemical hoses; wire sheathing; window profiles; vehicle-construction components, for example bodywork constituents, vibration dampers for engines; tires; furniture, for example chairs, tables, or shelving; cushion foam and mattress foam; tarpaulins, for example truck tarpaulins or tenting; gaskets; composite foils, such as foils for laminated safety glass, in particular for vehicle windows and for window panes; recording disks; synthetic leather; packaging containers; adhesive-tape foils, coatings, computer housings, and housings of electrical devices, for example kitchen machines.

The molding composition of the invention is also suitable for producing moldings and foils which come directly into contact with people or with foods. These are primarily medical products, hygiene products, packaging for food or drink, products for the interior sector, toys and child-care items, sports and leisure products, apparel, and also fibers for textiles, and the like.

The medical products which can be produced from the molding composition of the invention are for example tubes for enteral nutrition and hemodialysis, breathing tubes, infusion tubes, infusion bags, blood bags, catheters, tracheal tubes, gloves, breathing masks, or disposal syringes.

The packaging that can be produced from the molding composition of the invention for food or drink are for example freshness-retention foils, food-or-drink hoses, drinking-water hoses, containers for storing or freezing food or drink, lid gaskets, closure caps, crown corks, or synthetic corks for wine.

The products which can be produced from the molding composition of the invention for the interior sector are for example floorcoverings, which can have homogeneous structure or a structure composed of a plurality of layers, composed of at least one foamed layer, examples being sports floors and other floorcoverings, luxury vinyl tiles (LVT), synthetic leather, wailcoverings, or foamed or unfoamed wallpapers in buildings, or are cladding or console covers in vehicles.

The toys and child-care items which can be produced from the molding composition of the invention are for example dolls, inflatable toys, such as balls, toy figures, modeling clays, swimming aids, stroller covers, baby-changing mats, bedwarmers, teething rings, or bottles.

The sports and leisure products that can be produced from the molding composition of the invention are for example gymnastics balls, exercise mats, seat cushions, massage balls and massage rolls, shoes and shoe soles, balls, air mattresses, and drinking bottles.

The apparel that can be produced from the molding compositions of the invention is for example latex clothing, protective apparel, rain jackets, or rubber boots.

Non-PVC Applications:

The present invention also includes the use of the compounds of the invention as and/or in auxiliaries selected from: calendering auxiliaries; rheology auxiliaries; surfactant compositions, such as flow aids and film-forming aids, defoamers, antifoams, wetting agents, coalescing agents, and emulsifiers; lubricants, such as lubricating oils, lubricating greases, and lubricating pastes; quenchers for chemical reactions; phlegmatizing agents; pharmaceutical products; plasticizers in adhesives; impact modifiers and antiflow additives.

The figures described below and the examples provide further explanation of the invention. These figures and examples are not to be understood as restricting the invention.

The following abbreviations are used in the examples and figures below:

2,5-FDCA for 2,5-furandicarboxylic acid,

2,5-THFDCA for 2,5-tetrahydrofurandicarboxylic acid,

DMAP for 4-dimethylaminopyridine,

MTBE for tert-butyl methyl ether,

THF for tetrahydrofuran,

phr for parts by weight per 100 parts by weight of polymer.

### DESCRIPTION OF FIGURES

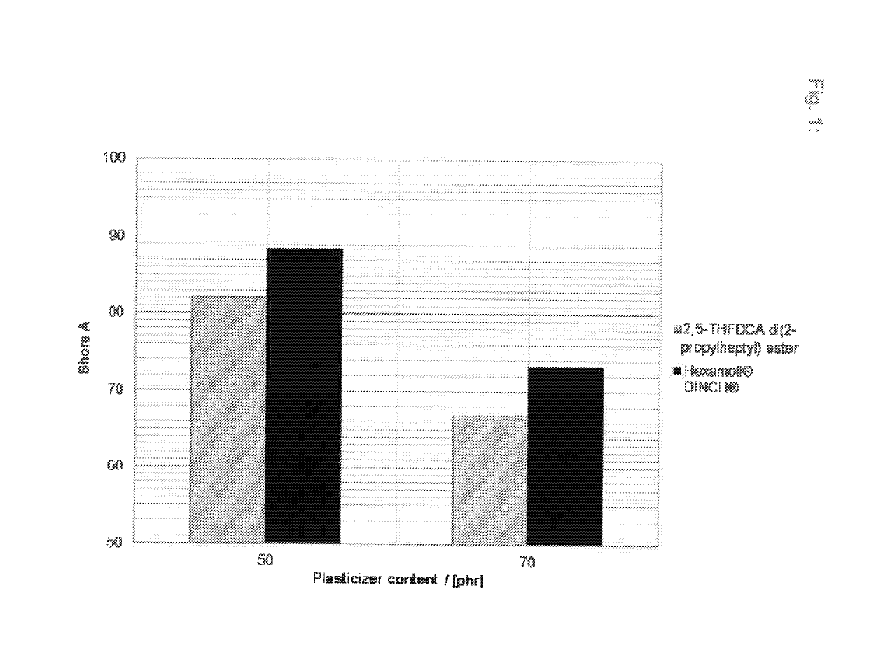

FIG. 1:

FIG. 1 shows, in the form of a bar chart, the Shore A hardness of flexible PVC test specimens which comprise different amounts of the plasticizer 2,5-THFDCA di(2-propylheptyl) ester (white hatched) and, as comparison, the commercially available plasticizer Hexamoll® DINCH® (black). The Shore A hardness has been plotted against the plasticizer content of the flexible PVC test specimens (stated in phr). The values measured were always determined after a time of 15 seconds.

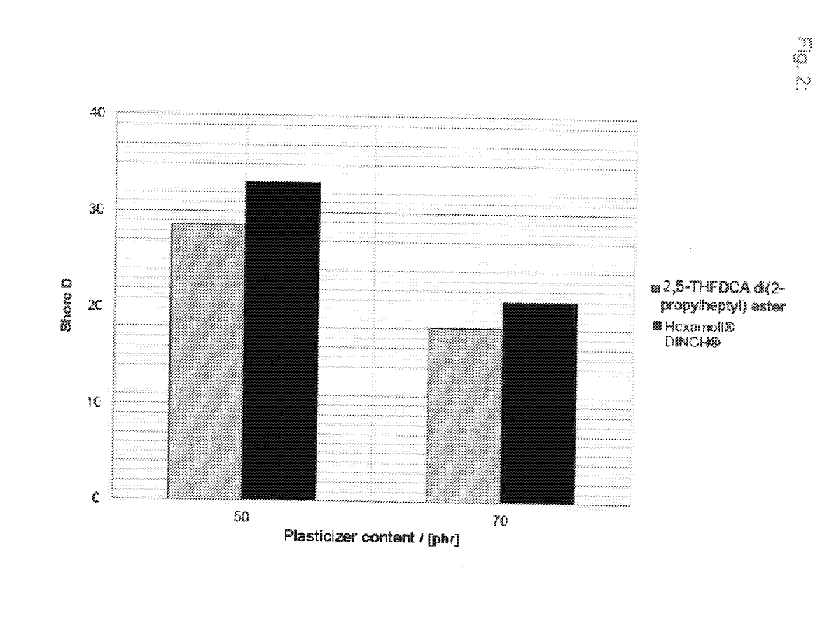

FIG. 2:

FIG. 2 shows, in the form of a bar chart, the Shore D hardness of flexible PVC test specimens which comprise 50 and, respectively, 70 phr of the plasticizer 2,5-THFDCA di(2-propylheptyl) ester of the invention (white hatched) and, as comparison, the commercially available plasticizer Hexamoll® DINCH® (black). The Shore D hardness has been plotted against the plasticizer content of the flexible PVC test specimens (stated in phr). The values measured were always determined after a time of 15 seconds.

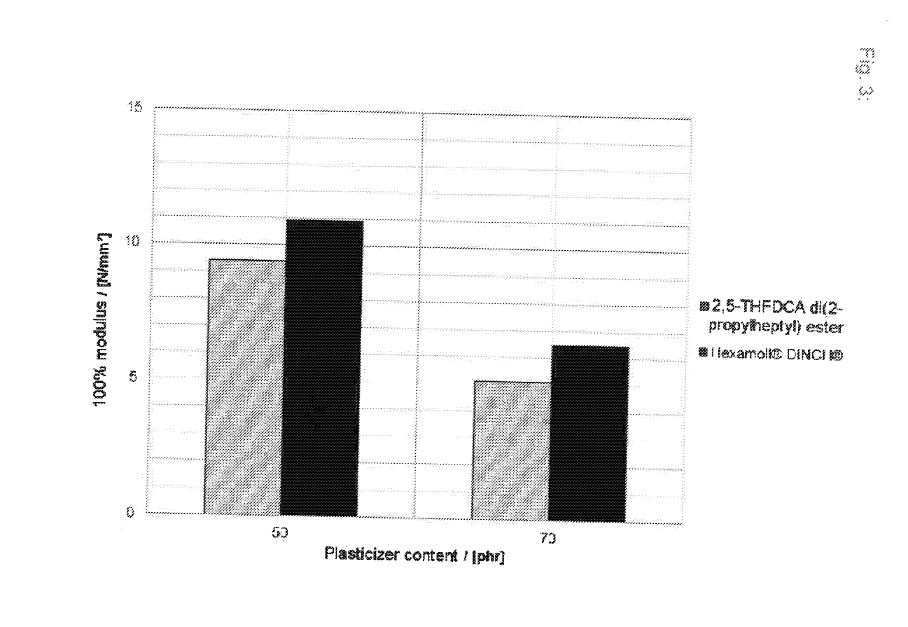

FIG. 3:

FIG. 3 shows, in the form of a bar chart, the 100% modulus of flexible PVC test specimens which comprise 50 and, respectively, 70 phr of the plasticizer 2,5-THFDCA di(2-propylheptyl) ester of the invention (white hatched) and, as comparison, the commercially available plasticizer Hexamoll® DINCH® (black). The 100% modulus has been plotted against the plasticizer content of the flexible PVC test specimens (stated in phr).

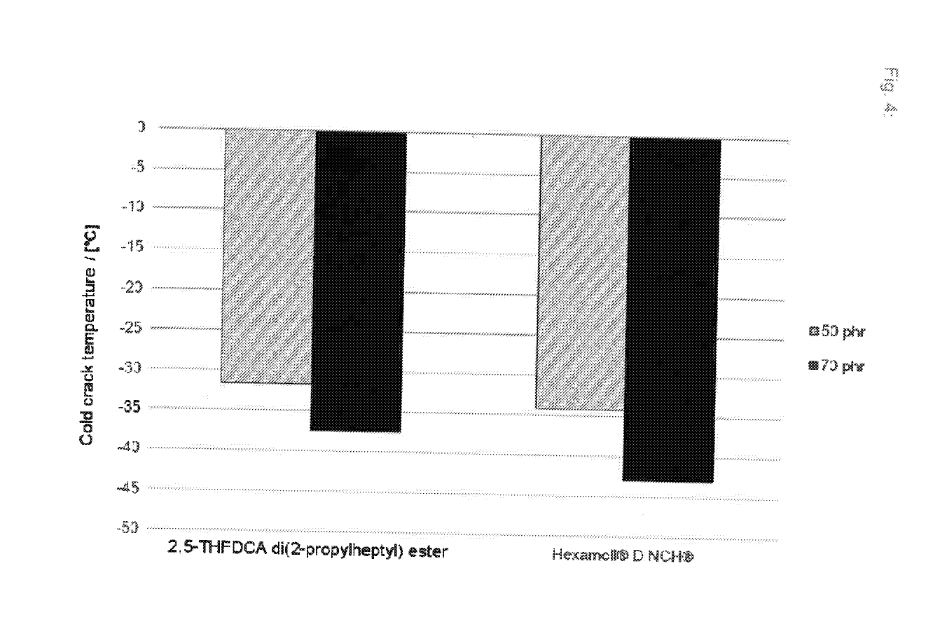

FIG. 4:

FIG. 4 shows, in the form of a bar chart, the cold crack temperature of flexible PVC foils which comprise the plasticizer 2,5-THFDCA di(2-propylheptyl) ester of the invention and, as comparison, the commercially available plasticizer Hexamoll® DINCH®. The chart shows the cold crack temperature in ° C. for flexible PVC foils with plasticizer content of 50 and 70 phr.

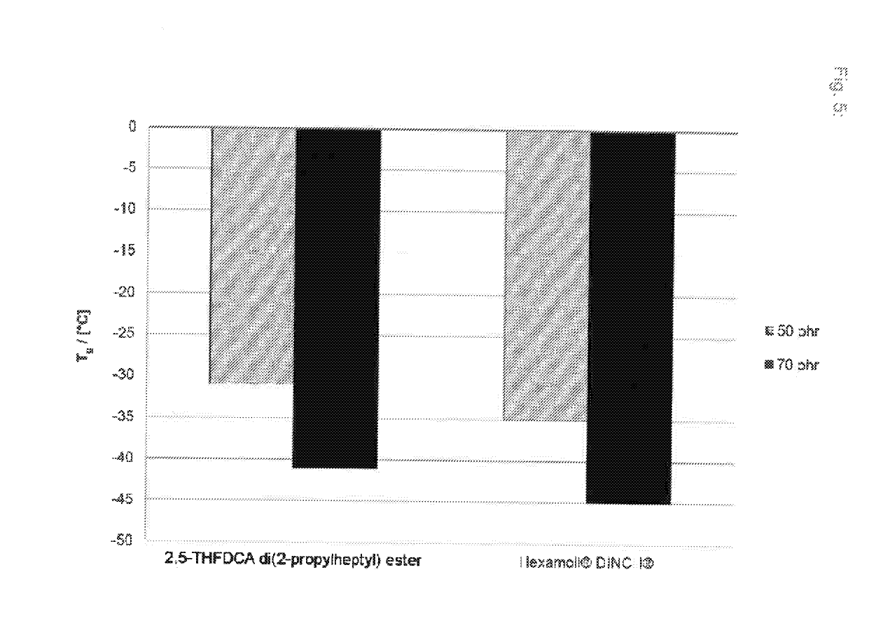

FIG. 5:

FIG. 5 shows, in the form of a bar chart, the glass transition temperature (T_{g}) of flexible PVC foils which comprise the plasticizer 2,5-THFDCA di(2-propylheptyl) ester of the invention and, as comparison, the commercially available plasticizer Hexamoll® DINCH®. The chart shows the glass transition temperature (T_{g}) in ° C. for flexible PVC foils with plasticizer content of 50 and 70 phr.

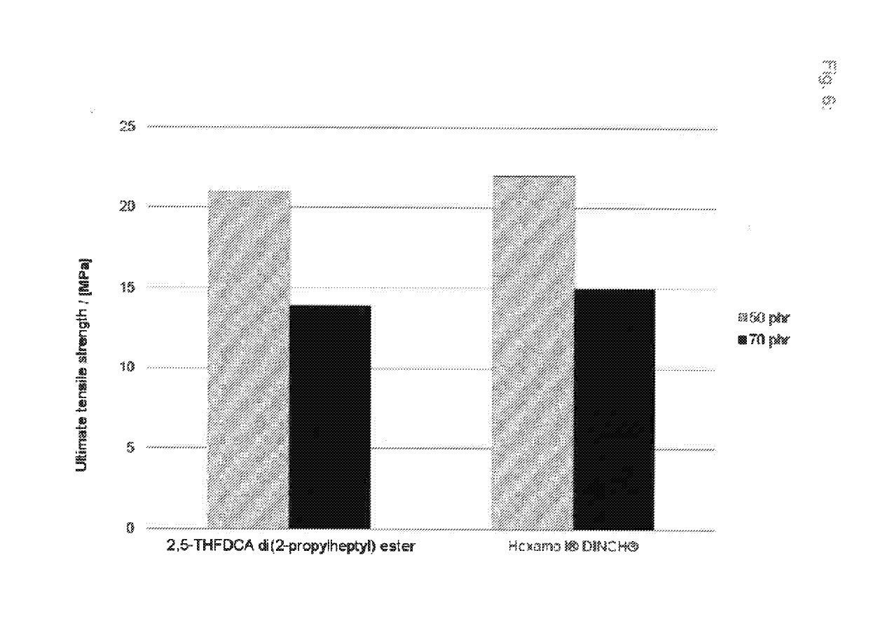

FIG. 6:

FIG. 6 shows, in the form of a bar chart, the ultimate tensile strength of flexible PVC foils which comprise the plasticizer 2,5-THFDCA di(2-propylheptyl) ester of the invention and, as comparison, the commercially available plasticizer Hexamoll® DINCH®. The chart shows the ultimate tensile strength in MPa for flexible PVC foils with plasticizer content of 50 and 70 phr.

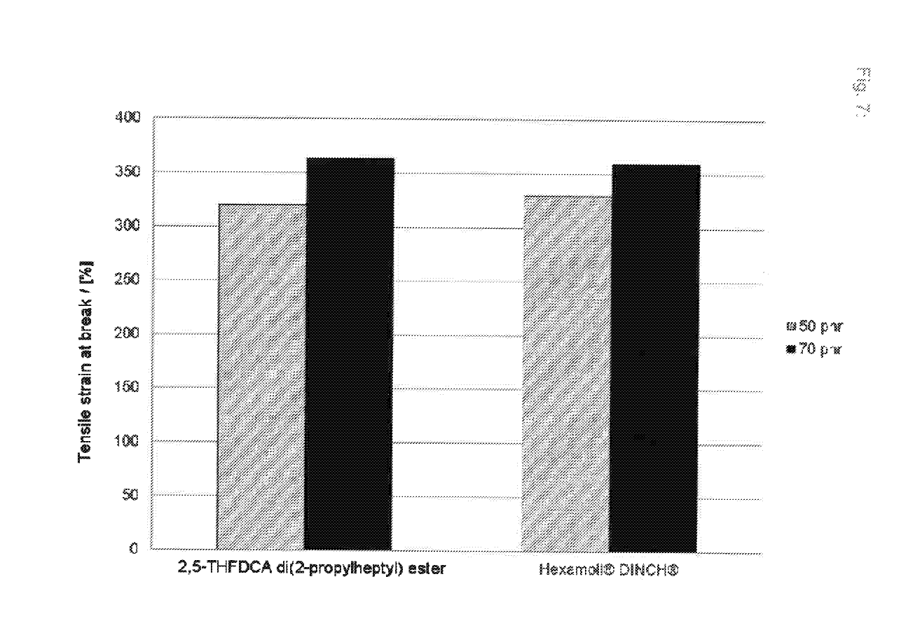

FIG. 7:

FIG. 7 shows, in the form of a bar chart, the tensile strain at break of flexible PVC foils which comprise the plasticizer 2,5-THFDCA di(2-propylheptyl) ester of the invention and, as comparison, the commercially available plasticizer Hexamoll® DINCH®. The chart shows the tensile strain at break in % of the initial value (=100%) for flexible PVC foils with plasticizer content of 50 and 70 phr.

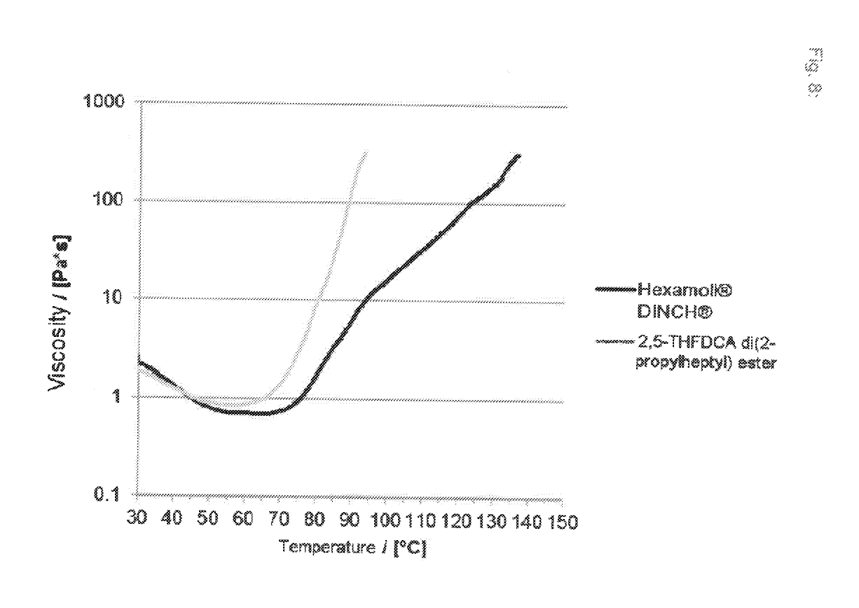

FIG. 8:

FIG. 8 shows the gelling behavior of PVC plastisols which comprise the plasticizer 2,5-THFDCA di(2-propylheptyl) ester of the invention and, as comparison, the commercially available plasticizer Hexamoll® DINCH®. The viscosity of the plastisols is shown as a function of temperature.

### EXAMPLES

### I) Production Examples

### Example 1

### Synthesis of di(2-propylhexyl) 2,5-tetrahydrofurandicarboxylate from dimethyl 2,5-furandicarboxylate via transesterification and hydrogenation

### Example 1.1

### Production of dimethyl 2,5-furandicarboxylate (=Step a)

3.30 kg of methanol were used as initial charge together with 0.10 kg of concentrated sulfuric acid in a 10 L glass reactor equipped with heating jacket, reflux condenser, and mechanical stirrer. 1.6 kg of 2,5-furandicarboxylic acid (2,5-FDCA) were slowly added to this mixture, with vigorous stirring. The dense white suspension that forms was then heated to 70° C. (reflux). The course of the reaction was monitored by means of HPLC analysis, whereupon after about 20 h a clear solution was obtained, with complete conversion of the 2,5-FDCA. The reaction mixture was then cooled to 65° C., and neutralized with saturated NaHCO₃ solution and solid NaHCO₃ (pH 7). During the neutralization, a dense white suspension again formed, and was cooled to 10° C., stirred for a further 0.5 h, and then filtered by way of a P2 sintered glass frit. The filtercake was washed three times with 1 L of cold water, whereupon about 2 kg of wet solid was obtained.

For purification and recrystallization, the wet solid was added to 6.00 kg of 2-butanone in a 10 L glass reactor equipped with heating jacket, reflux condenser, and mechanical stirrer. The suspension was heated to 70° C., whereupon a clear solution was obtained. 1.00 kg of water was then added, and this led to formation of a brownish orange aqueous phase. It was sometimes necessary to add 900 mL of saturated sodium chloride solution in order to achieve phase separation. The aqueous phase was removed, and the organic phase was cooled to 20° C., without stirring, whereupon the crystallization of the product began (usually at about 35° C.). The crystalline suspension was then cooled to 0° C. and stirred overnight. The suspension was then filtered by way of a P2 sintered glass frit, and the filtercake was washed with 1 L of cold methanol. The solid residue was dried at room temperature in vacuo. The desired dimethyl 2,5-furandicarboxylate was obtained in a yield of from 50 to 60% and in a purity of >99%. The identity and purity of the final product was determined by means of NMR and HPLC (HPLC column: Varian Polaris 3μ C₁₈-A, 150×4.6 mm).

### Example 1.2

### Catalytic Hydrogenation (=Step b2)

A 20% by weight solution of dimethyl 2,5-furandicarboxylate in THF was charged to a nitrogen-filled 2.5 L Hastelloy C autoclave from Parr Instrument, equipped with a mechanical stirrer with magnetic coupling, thermocouple, sampling tube, and baffles. 120 g of a heterogeneous Pd/Pt catalyst (0.4% by weight of Pd/0.4% by weight of Pt on ZrO₂, produced by analogy with DE4429014, example 6) were then added, and the nitrogen atmosphere was replaced by a hydrogen atmosphere by filling and ventilating the autoclave with hydrogen three times. The final pressure of hydrogen was increased to 200 bar, and the autoclave was heated to 180° C. The progress of the reaction was monitored by means of GC analysis. After complete conversion (usually after from 40 to 60 hours), the autoclave was cooled and ventilated, and the contents were filtered in order to remove the solid catalyst. The solvent in the filtrate was then removed by distillation under reduced pressure, and the retained crude product was diluted in 300 mL of tert-butyl methyl ether and transferred to a separating funnel. The organic phase was washed twice with saturated NaHCO₃ solution and once with saturated sodium chloride solution. The solvent and other volatile constituents were then removed by distillation under reduced pressure. The crude products were purified by fractional distillation, whereupon dimethyl 2,5-tetrahydrofurandicarboxylate was obtained in the form of colorless to brownish, viscous liquid. The desired dimethyl 2,5-tetrahydrofurandicarboxylate was obtained here in a yield of 57% and in a purity of 98.2%. The identity and purity of the final product were determined by means of NMR and GC-MS analysis (GC column: Agilent J&W DB-5, 30 m×0.32 mm×1.0 μm).

### Example 1.3

### Transesterification of dimethyl 2,5-tetrahydrofurandicarboxylate (=Step c2)

204 g (1.08 mol, 1.0 equivalent) of dimethyl 2,5-tetrahydrofurandicarboxylate were dissolved in 200 g of n-heptane in a 2 L round-necked flask equipped with a dropping funnel with pressure equalization, and 693 g (4.38 mol, 4.0 equivalents) of 2-propyl-1-heptanol, and also a mixed titanium(IV) propoxide/butoxide complex (3 mol % of titanium) were added. The mixture was heated to reflux (from 100 to 126° C.) for 22 hours, with stirring. The course of the reaction was monitored by means of GC analysis. After complete conversion, the reaction mixture was cooled to room temperature and filtered, and the titanium(IV) alkoxide was hydrolyzed via addition of 100 mL of water. The two-phase mixture was transferred to a separating funnel, the aqueous phase was removed, and the organic phase was washed once with saturated sodium chloride solution. The solvent and other volatile constituents were then removed by distillation under reduced pressure. The crude product was purified by means of fractional distillation, whereupon di(2-propylheptyl) 2,5-tetrahydrofuran dicarboxylate was obtained in the form of clear colorless liquid in a yield of 58% and in a purity of 98.5%. The identity and purity of the final product was determined by means of NMR and GC-MS analysis (GC column: Agilent J&W DB-5, 30 m×0.32 mm×1.0 μm).

### Example 2

### Synthesis of di(2-propylheptyl) 2,5-tetrahydrofurandicarboxylate Via Direct Esterification and Hydrogenation

### Example 2.1

### Production of di(2-propylheptyl) 2,5-furandicarboxylate (=Step b1)

949 g (6.00 mol, 4.0 equivalents) of 2-propyl-1-heptanol in 500 g of toluene and 234 g (1.50 mol, 1.0 equivalent) of 2,5-furandicarboxylic acid were used as initial charge in a 2 L round-necked flask equipped with a Dean-Stark water separator and a dropping funnel with pressure equalization. The mixture was heated to reflux, with stirring, and 11.5 g (0.12 mol, 8 mol %) of 99.9% sulfuric acid were added in from 3 to 4 portions whenever the reaction slowed. The course of the reaction was monitored on the basis of the amount of water separated in the Dean-Stark apparatus. After complete conversion, a specimen was taken from the reaction mixture and analyzed by GC. The reaction mixture was cooled to room temperature, transferred to a separating funnel, and washed twice with saturated NaHCO₃ solution. The organic phase was washed with saturated sodium chloride solution and dried with anhydrous Na₂SO₄, and the solvent was removed under reduced pressure. The crude product was purified by means of fractional distillation. The desired di(2-propylheptyl) 2,5-furandicarboxylate was obtained here in a yield of 58% and in a purity of 97.8%. The identity and purity of the final product was determined by means of NMR and GC-MS analysis (GC column: Agilent J&W DB-5, 30 m×0.32 mm×1.0 μm or Ohio Valley OV-1701 60 m×0.32 mm×0.25 μm).

Catalytic Hydrogenation (=Step c1):

A 20% by weight solution of di(2-propylheptyl) 2,5-furandicarboxylate in THF was charged to a nitrogen-filled 2.5 L Hastelloy C autoclave from Parr Instrument, equipped with a mechanical stirrer with magnetic coupling, thermocouple, sampling tube, and baffles. 120 g of a heterogeneous Pd/Pt catalyst (0.4% by weight of Pd/0.4% by weight of Pt on ZrO₂, produced by analogy with DE 4429014, example 6) were then added, and the nitrogen atmosphere was replaced three times with hydrogen at superatmospheric pressure. The final pressure of hydrogen was increased to 200 bar, and the autoclave was heated to 180° C. The progress of the reaction was monitored by means of GC analysis. After complete conversion (usually after from 40 to 60 hours), the autoclave was ventilated, and the contents were filtered in order to remove the solid catalyst. The solvent in the filtrate was then removed by distillation under reduced pressure, and the retained crude product was diluted in 300 mL of MTBE and transferred to a separating funnel. The organic phase was washed twice with saturated NaHCO₃ solution and once with saturated sodium chloride solution. The solvent and other volatile constituents were then removed by distillation under reduced pressure. The crude product was purified by fractional distillation, whereupon di(2-propylheptyl) 2,5-tetrahydrofurandicarboxylate was obtained in the form of colorless to brownish, viscous liquid in a yield of 53% and in a purity of 95.9%. The identity and purity of the final product were determined by means of NMR and GC-MS analysis (GC column: Agilent J&W DB-5, 30 m×0.32 mm×1.0 μm).

### Example 3

### Synthesis of di(2-ethylhexyl) 2,5-tetrahydrofurandicarboxylate

Di(2-ethylhexyl) 2,5-tetrahydrofurandicarboxylate was synthesized by analogy with example 2 (steps b1 and c1). Distillative purification gave di(2-ethylhexyl) 2,5-tetrahydrofurandicarboxylate as colorless to brownish liquid in a yield of 31% and in a purity of 99.5%. The identity and purity of the final product were determined by means of NMR and GC-MS analysis (GC column: Agilent J&W DB-5, 30 m×0.32 mm×1.0 μm).

### Example 4

### Synthesis of di(n-octyl) 2,5-tetrahydrofurandicarboxylate

Di(n-octyl) 2,5-tetrahydrofurandicarboxylate was synthesized by analogy with example 2 (steps b1 and c1). Distillative purification gave di(n-octyl) 2,5-tetrahydrofurandicarboxylate as colorless to brownish liquid in a yield of 45% and in a purity of 98.7%. The identity and purity of the final product were determined by means of NMR and GC-MS analysis (GC column: Agilent J&W DB-5, 30 m×0.32 mm×1.0 μm).

### Example 5

### Synthesis of the di-2-propylheptyl ether of 2,5-di(hydroxymethyl)tetrahydrofuran

10.6 g of 2,5-di(hydroxymethyl)tetrahydrofuran (80 mmol, 1.0 equivalent) were dissolved in 140 ml of toluene in a 500 mL four-necked flask equipped with a mechanical stirrer, dropping funnel, thermometer, and reflux condenser. 22.4 g (400 mmol, 5.0 equivalents) of powdered KOH were added in portions to this mixture at room temperature over a period of 30 minutes and with continuous stirring. The mixture was then stirred at reflux for from 3 to 4 hours. 60.0 g of molecular sieve (3 Å) were then added, and the mixture was stirred at reflux for a further hour, whereupon a cream-colored suspension was obtained. The mixture was cooled to 90° C., and 46.0 g (208 mmol, 2.6 equivalents) of 4-(bromomethyl)nonane dissolved in 40 mL of toluene were added dropwise over 1.5 hours. The dropping funnel was washed with 20 mL of toluene, and the wash solution was combined with the reaction mixture. The course of the reaction was monitored by means of GC analysis. After the end of the reaction, (usually from 40 to 80 hours) the mixture was cooled to room temperature. The glass containers were washed with MTBE, the washing solution was combined with the reaction mixture, and the resultant white suspension was filtered. The salt residues removed by filtration were washed with MTBE. The combined organic phases were in each case washed in succession once with saturated sodium chloride solution, with saturated ammonium chloride solution, and again with saturated sodium chloride solution, and finally dried over Na₂SO₄. The solvent and other volatile constituents were then removed by distillation under reduced pressure, and the residue was dried under high vacuum. The crude product was purified by means of fractional distillation, whereupon the di-2-propylheptyl ether of 2,5-di(hydroxymethyl)tetrahydrofuran was obtained in the form of clear colorless liquid in a yield of 38% and in a purity of 82%. The identity and purity of the final product were determined by means of NMR and GC-MS analysis (GC column: Agilent J&W DB-5, 30 m×0.32 mm×1.0 μm).

### Example 6

### Synthesis of 2,5-di(hydroxymethyl)tetrahydrofuran diethylhexanoate

39.6 g of 2,5-di(hydroxymethyl)tetrahydrofuran (300 mmol, 1.0 equivalent), 91.1 g of triethylamine (900 mmol, 3.0 equivalents) and 3.70 g of DMAP (30.0 mmol, 0.1 equivalent) were dissolved in 700 ml of THF in a 1 L round-necked flask equipped with a mechanical stirrer, dropping funnel with pressure equalization, thermometer, and reflux condenser. 103 g (633 mmol, 2.1 equivalents) of 2-ethylhexanoyl chloride were added dropwise to this mixture over a period of one hour, with continuous stirring. During the addition of the acyl chloride, the reaction temperature increased and was optionally maintained at from 20 to 30° C. via cooling with an ice bath. Once addition had ended, the reaction mixture was stirred for one hour at room temperature and for four hours at 60° C. The mixture was then cooled to room temperature and stirred overnight. The course of the reaction was monitored by means of GC analysis. After the end of the reaction, the reaction mixture was transferred to a separating funnel and washed with 100 mL of water. The aqueous phase was extracted three times with 150 mL of ethyl acetate. The combined organic phases were washed with saturated sodium chloride solution and dried over Na₂SO₄. The solvent and other volatile constituents were then removed by distillation under reduced pressure. The crude product was purified by means of fractional distillation, whereupon 53.5 g (150 mmol) of 2,5-di(hydroxymethyl)tetrahydrofuran diethylhexanoate were obtained in the form of clear yellow liquid in a yield of 50% and in a purity of 99.9%. The identity and purity of the final product were determined by means of NMR and GC-MS analysis (GC column: Agilent J&W DB-5, 30 m×0.32 mm×1.0 μm).

II) Production of Plasticized PVC Foils on a Roll Mill and of PVC Test Specimens:

II.a) Production of PVC Foils on a Roll Mill:

To assess the plasticizing properties of the plasticizers of the invention and of the comparative compounds during thermoplastic processing, flexible PVC foils of thickness 0.5 mm were produced. These foils were produced via rolling and pressing of plasticized PVC.

In order to eliminate effects due to different additives, the formulation below was used in each case for producing the plasticized PVC:

| Additive | phr |
|---|---|
| Solvin 271 SP¹⁾ | 100 |
| Plasticizer | 50 and, respectively, 70 |
| SLX 781²⁾ reagent | 2 |

¹⁾commercially obtainable PVC from Solvin GmbH & Co. KG, produced via suspension polymerization (K value in accordance with ISO 1628-2: 71)
²⁾liquid Ba—Zn stabilizer from Reagens Deutschland GmbH

The ingredients were mixed at room temperature with a manual mixer. The mixture was then plastified in a steam-heated laboratory mixing unit from Collin (150) and processed to give a milled sheet. The rotation rates were 15 rotations/minute (front roll) and 12 rotations/minute (rear roll), and the roll-milling time was 5 minutes. This gave a milled sheet of thickness 0.55 mm. The cooled milled sheet was then pressed in a 400 P Collin press within a period of 400 seconds under a pressure of 220 bar to give a flexible PVC foil of thickness 0.50 mm.

The respective conditions for the roll mill and press can be found in the table below:

| | | Plasticizer | Roll- | |
|---|---|---|---|---|
| | | content | milling | Pressing |
| Ex. No. | Product | [phr] | [° C.] | [° C.] |
| 1 | 2,5-THFDCA di(2-propylheptyl) | 50/70 | 180/170 | 190/180 |
| | ester | | | |
| comp 1 | Hexamoll ® DINCH ®³⁾ | 50/70 | 180/175 | 190/185 |
| comp 2 | 2,5-FDCA di(2-propylheptyl) | 70 | 170 | 180 |
| | ester | | | |

³⁾Diisononyl cyclohexanedicarboxylate from BASF SE (CAS No. in Europe and Asia: 166412-78-8; CAS No. in the USA: 474919-59-0)

The test specimens needed for the tests were produced from the resultant roll-milled and pressed foils.

II.b) Production of Test Specimens:

The test specimens with dimensions 49 mm×49 mm×10 mm (length×width×thickness) were produced via pressing from roll-milled foils at a temperature which was 10° C. above the roll-milling temperature. For the performance tests, the test specimens were aged for 7 days at 23° C.+/−1.0° C. and 50%+/−5% relative humidity (to DIN EN ISO 291).

III) Performance Tests:

III.a) Determination of Solvation Temperature in Accordance with DIN 53408:

To characterize the gelling performance of the plasticizers of the invention in PVC, solvation temperature was determined in accordance with DIN 53408. In accordance with DIN 53408, a droplet of a slurry of 1 g of PVC in 19 g of plasticizer is observed in transmitted light under a microscope equipped with a heatable stage. The temperature here is increased linearly by 2° C. per minute, starting at 60° C. The solvation temperature is the temperature at which the PVC particles become invisible, i.e. it is no longer possible to discern their outlines and contrasts. The lower the solvation temperature, the better the gelling performance of the relevant substance for PVC.

The table below lists the solvation temperatures of the di(n-octyl) 2,5-tetrahydrofurandicarboxylate, di(2-ethylhexyl) 2,5-tetrahydrofurandicarboxylate, and di(2-propylheptyl) 2,5-tetrahydrofurandicarboxylate plasticizers of the invention and, as comparison, of Hexamoll® DINCH® (comp 1) and the corresponding diesters of furandicarboxylic acid (comp 2 to comp 4) or of phthalic acid (comp 5 to comp 7).

| | | Solvation temperature |
|---|---|---|
| | | in accordance with |
| Ex. No. | Substance | DIN 53408 [° C.] |
| 1 | Di(2-propylheptyl) 2,5- | 127 |
| | tetrahydrofurandicarboxylate | |
| 2 | Di(2-ethylhexyl) 2,5- | 110 |
| | tetrahydrofurandicarboxylate | |
| 3 | Di(n-octyl) 2,5- | 105 |
| | tetrahydrofurandicarboxylate | |
| Comp 1 | Hexamoll ® DINCH ®³⁾ | 151 |
| Comp 2 | Di(2-propylheptyl) 2,5- | 137 |
| | furandicarboxylate | |
| Comp 3 | Di(2-ethylhexyl) 2,5-furandicarboxylate | 118 |
| Comp 4 | Di(n-octyl) 2,5-furandicarboxylate | 118 |
| Comp 5 | Di(2-propylheptyl) phthalate⁴⁾ | 146 |
| Comp 6 | Di(isononyl) phthalate⁵⁾ | 132 |
| Comp 7 | Di(2-ethylhexyl) phthalate⁶⁾ | 124 |

³⁾Diisononyl cyclohexanedicarboxylate from BASF SE (CAS No. in Europe and Asia: 166412-78-8; CAS No. in the USA: 474919-59-0)
⁴⁾Di(2-propylheptyl) benzene-1,2-dicarboxylate (CAS No. 53306-54-0)
⁵⁾Di(isononyl) benzene-1,2-dicarboxylate (CAS No. 28553-12-0 or 68515-48-0)
⁶⁾Di(2-ethylhexyl) benzene-1,2-dicarboxylate (CAS No. 117-81-7)

As can be seen from the table, the plasticizers of the invention exhibit lower solvation temperatures than Hexamoll® DINCH® (comp 1). Their solvation temperatures are also lower than those of the corresponding diesters of furandicarboxylic acid (comp 2 to comp 4) or the corresponding diesters of phthalic acid (comp 5 to comp 7).

III.b) Physical Properties:

The table below lists the most significant physical properties of di(2-propylheptyl) 2,5-tetrahydrofurandicarboxylate (example 1) in comparison with the Hexamoll® DINCH® plasticizer used in the market (comparative example comp 1).

Plasticizer:

| | Di(2-propylheptyl) 2,5- | Hexamoll ® |
|---|---|---|
| | tetrahydrofuran-dicarboxylate | DINCH ® |
| Density (20° C.) | 0.958 | 0.944-0.954 |
| [g/cm³] | | |
| Viscosity (20° C.) | 40 | 44-60 |
| [mPa · s] | | |

Relevant physical properties for the plasticizer application alongside the solvation temperature in accordance with DIN 53408 are specifically density and viscosity. In comparison with the plasticizer Hexamoll® DINCH®, which is commercially available and regarded as having advantageous properties, the di(2-propylheptyl) 2,5-tetrahydrofurandicarboxylate plasticizer of the invention actually exhibits slightly lower, and therefore more advantageous, viscosity with comparable density.

III.c) Shore Hardness Determination:

Shore A and D hardness were determined in accordance with DIN EN ISO 868 with a DD-3 digital durometer from Hildebrand. The test specimens were produced as in example II.c). The values shown in FIG. 1 and FIG. 2 are in each case the average value from 20 measurements per test specimen (10 measurements on the front side and 10 measurements on the reverse side). The value measured was always determined after a time of 15 seconds.

As can be seen from the charts of FIG. 1 and FIG. 2, 2,5-THFDCA dibutyl ester of the invention exhibits slightly better plasticizing effect than the commercially available plasticizer Hexamoll® DINCH®.

III.d) Determination of 100% Modulus:

100% modulus is another property, alongside Shore hardness, that characterizes the plasticizing effects of plasticizers, i.e. plasticizer efficiency.

100% modulus was determined in accordance with DIN EN ISO 527 part 1 and 3 with a TMZ 2.5/TH1S tester from Zwick. The test specimens of dimensions 150 mm×10 mm×0.5 mm (length×width×thickness) correspond to type 2 in accordance with DIN EN ISO 527 part 3, and are punched out from the rolled/pressed foils by means of a hole punch. The test specimens are conditioned for 7 days before the test. The conditioning and the tensile tests take place at 23° C.+/−1.0° C. and 50%+/−5% relative humidity in accordance with DIN EN ISO 291. The values plotted in FIG. 3 are in each case average values from the testing of 10 individual test specimens.

As can be seen from the chart of FIG. 3, 2,5-THFDCA di(2-propylheptyl) ester of the invention exhibits better plasticizing effect than the commercially available plasticizer Hexamoll® DINCH®.

III.e) Determination of Low-Temperature Flexibility:

To determine low-temperature flexibility, PVC foils were used which comprised different concentrations of the respective plasticizer to be tested. Two methods were used. Firstly, cold crack temperature was determined by a method based on the standard DIN 53372, which is no longer current, and secondly the glass transition temperature T_{g} of the foils was determined by means of DMA (dynamic mechanical analysis) in accordance with ISO 6721-7 from the maximum of the loss modulus “G”. FIGS. 4 and 5 show the results from the two test methods.

As is apparent from the charts of FIGS. 4 and 5, the PVC foils which comprise 2,5-THFDCA di(2-propylheptyl) ester of the invention exhibit a slightly increased cold crack temperature in comparison with PVC foils using Hexamoll® DINCH®. The same applies to the glass transition temperature.

III.f) Determination of Ultimate Tensile Strength and of Tensile Strain at Break:

Ultimate tensile strength and tensile strain at break were determined in accordance with DIN EN ISO 527 part 1 and 3 with a TMZ 2.5/TH1S tester from Zwick. The test specimens used with dimensions 150 mm×10 mm×0.5 mm (length×width×thickness) correspond to type 2 in accordance with DIN EN ISO 527 part 3, and were conditioned for 7 days prior to testing. The conditioning and the tensile tests took place at 23° C.+/−1.0° C. and relative humidity of 50%+/−5% in accordance with DIN EN ISO 291.

The values indicated in FIGS. 6 and 7 are in each case average values from the testing of 10 individual test specimens.

As is apparent from FIGS. 6 and 7, when the PVC test specimens with the 2,5-THFDCA 2-propylheptyl ester plasticizer of the invention are compared with the test specimens with the commercially available plasticizer Hexamoll® DINCH®, they exhibit identical or only slightly lower values for ultimate tensile strength and for tensile strain at break.

III.g) Determination of Gelling Behavior of PVC Plastisols:

In order to study the gelling behavior of PVC plastisols based on the plasticizers of the invention, PVC plastisols with the 2,5-THFDCA di(2-propylheptyl) ester plasticizer and with the commercially available Hexamoll® DINCH® plasticizer were produced in accordance with the following formulation:

| Additive | phr |
|---|---|
| Solvin 372 NF⁷⁾ | 100 |
| Plasticizer | 60 |
| Reagens SLX 781⁸⁾ | 2 |

⁷⁾commercially available PVC from Solvin GmbH & Co. KG, produced via suspension polymerization (K value in accordance with ISO 1628-2: 73)
⁸⁾liquid Ba—Zn stabilizer from Reagens Deutschland GmbH

The plastisols were produced by using a dissolver, with stirring at about 800 revolutions/minute, to add the PVC to the weighed mixture of plasticizer and heat stabilizer. Once PVC addition had ended, the mixture was homogenized at 2500 revolutions/minute for 2.5 minutes, and then deaerated in vacuo in a desiccator.

In order to gel a liquid PVC plastisol and to convert it from the condition of PVC particles homogeneously dispersed in plasticizer to a homogeneous, solid flexible PVC matrix, the energy necessary for this purpose has to be introduced in the form of heat. The processing parameters available for this purpose comprised temperature and residence time. The faster the gelling process (indicator here being the solvation temperature, i.e. the lower this is, the faster is the gelling of the material), the lower the level that can be selected for temperature (at the same residence time) or for residence time (at the same temperature).

The gelling behavior of a plastisol is studied by an internal method with an MCR101 rheometer from Anton Paar. The viscosity of the paste is measured here during heating with constant shear (rotation). The measurement uses a plate-on-plate system (PP50) beginning at 30° C. with a shear rate of 10 1/s and with a heating rate of 5° C./minute.

The viscosity of a plastisol generally falls initially as temperature rises, and reaches a minimum. The viscosity then rises. The temperature at the minimum of the curve, and the steepness of the rise after the minimum, provide information about the gelling behavior, i.e. the lower the temperature at the minimum and the steeper the subsequent rise, the better or quicker the gelling.

As is very clear from FIG. 8, when the PVC plastisol with the 2,5-THFDCA 2-propylheptyl ester plasticizer of the invention is compared with the PVC plastisol with the commercially available Hexamoll® DINCH® plasticizer, it exhibits markedly quicker gelling.

1.-26. (canceled)

27. A compound of the general formula (I) 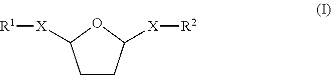
in which
X is *—(C═O)—O—, *—(CH₂)ₙ—O— or *—(CH₂)ₙ—O—(C═O)—, where * is the point of linkage to the tetrahydrofuran ring, and n has the value 0, 1, or 2;
and
R¹ and R² are selected mutually independently from n-octyl, 2-ethylhexyl, n-nonyl, isononyl, isodecyl, 2-propylheptyl, n-undecyl or isoundecyl.

28. The compound according to claim 27, where the definitions of R¹ and R² are identical.

29. The compound according to claim 27, where R¹ and R² are both 2-ethylhexyl, both isononyl or both 2-propylheptyl.

30. The compound according to claim 27, where both of the groups X are *—(C═O)—O—.

31. A plasticizer composition comprising at least one compound of the general formula (I) as defined in claim 27 and at least one plasticizer different from the compounds of the formula (I).

32. The plasticizer composition according to claim 31, where the plasticizer different from the compounds (I) is selected from the group consisting of dialkyl phthalates, alkyl aralkyl phthalates, dialkyl terephthalates, trialkyl trimellitates, dialkyl adipates, alkyl benzoates, dibenzoic esters of glycols, hydroxybenzoic esters, esters of saturated mono- and dicarboxylic acids, esters of unsaturated dicarboxylic acids, amides and esters of aromatic sulfonic acids, alkylsulfonic esters, glycerol esters, isosorbide esters, phosphoric esters, citric triesters, alkylpyrrolidone derivatives, 2,5-furandicarboxylic esters, 2,5-tetrahydrofurandicarboxylic esters different from compounds (I), epoxidized vegetable oils based on triglycerides and saturated or unsaturated fatty acids, polyesters derived from aliphatic and/or aromatic polycarboxylic acids with at least dihydric alcohols.

33. A molding composition comprising at least one polymer and at least one compound of the formula (I) as claimed in claim 27.

34. The molding composition according to claim 33, which further comprises at least one plasticizer different from the compounds of the general formula (I).

35. The molding composition according to claim 33, where the polymer involves a thermoplastic polymer selected from the group consisting of
homo- and copolymers which comprise at least one copolymerized monomer selected from C₂-C₁₀-monoolefins, 1,3-butadiene, 2-chloro-1,3-butadiene, vinyl alcohol and its C₂-C₁₀-alkyl esters, vinyl chloride, vinylidene chloride, vinylidene fluoride, tetrafluoroethylene, glycidyl acrylate, glycidyl methacrylate, acrylates and methacrylates of C₁-C₁₀-alcohols, vinylaromatics, (meth)acrylonitrile, maleic anhydride, and α,β-ethylenically unsaturated mono- and dicarboxylic acids,
homo- and copolymers of vinyl acetals,
polyvinyl esters,
polycarbonates,
polyesters,
polyethers,
polyether ketones,
thermoplastic polyurethanes,
polysulfides,
polysulfones,
polyether sulfones,
cellulose alkyl esters,
and mixtures thereof.

36. The molding composition according to claim 35, where the thermoplastic polymer is selected from polyvinyl chloride (PVC), polyvinyl butyral (PVB), homo- and copolymers of vinyl acetate, homo- and copolymers of styrene, polyacrylates, thermoplastic polyurethanes (TPUs), or polysulfides.

37. The molding composition according to claim 35, where the thermoplastic polymer involves polyvinyl chloride (PVC).

38. The molding composition according to claim 37, comprising at least one compound of the general formula (I) and optionally at least one plasticizer different from the compounds of the general formula (I), where the total plasticizer content is from 1.0 to 400 phr.

39. The molding composition according to claim 35, comprising at least one thermoplastic polymer different from polyvinyl chloride, at least one compound of the general formula (I), and optionally at least one plasticizer different from the compounds of the general formula (I), where the total plasticizer content is from 0.5 to 300 phr.

40. The molding composition according to claim 33, where the polymer is an elastomer.

41. The molding composition according to claim 40, comprising at least one compound of the general formula (I) and optionally at least one plasticizer different from the compounds of the general formula (I), where the total plasticizer content is from 1.0 to 60 phr.

42. A process for producing the compound according to claim 27 which comprises
a) optionally reacting 2,5-furandicarboxylic acid or an anhydride or acyl halide thereof with a C₁-C₃-alkanol in the presence of a catalyst to give a di(C₁-C₃-alkyl) 2,5-furandicarboxylate,
b1) reacting 2,5-furandicarboxylic acid or an anhydride or acyl halide thereof, or the di(C₁-C₃-alkyl) 2,5-furandicarboxylate obtained in step a), with at least one alcohol R¹—OH and, if R¹ and R² are different, also with at least one alcohol R²—OH, in the presence of at least one catalyst to give a compound of the formula (I.1a), 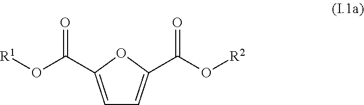
c1) hydrogenating the compound (I.1a) obtained in step b1) with hydrogen in the presence of at least one hydrogenation catalyst to give the compound of the general formula (I.1),
or
b2) hydrogenating the 2,5-furandicarboxylic acid or the di(C₁-C₃-alkyl) 2,5-furandicarboxylate obtained in step a) with hydrogen in the presence of at least one hydrogenation catalyst to give a compound of the general formula (I.1b), 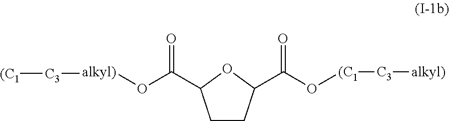
c2) reacting the compound (I.1b) obtained in step b2) with at least one alcohol R¹—OH and, if R¹ and R² are different, also with at least one alcohol R²—OH, in the presence of a catalyst to give a compound of the formula (I.1).

43. A process for producing compounds of the general formula (I.2) or (I.3), 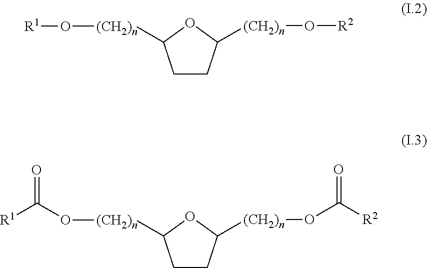
in which
R¹ and R² are selected mutually independently from n-octyl, 2-ethylhexyl, n-nonyl, isononyl, isodecyl, 2-propylheptyl, n-undecyl or isoundecyl,
and n has the value 1 or 2,
where
a) 2,5-di(hydroxymethyl)tetrahydrofuran (n=1) or for 2,5-di(hydroxyethyl)tetrahydrofuran (n=2), reaction is carried out with at least one alkylating reagent R¹—Z and, if R¹ and R² are different, also with at least one alkylating reagent R²—Z, where Z is a leaving group, in the presence of a base to give compounds of the formula (I.2),
or
b) 2,5-di(hydroxymethyl)tetrahydrofuran (n=1) or for 2,5-di(hydroxyethyl)tetrahydrofuran (n=2), reaction is carried out with at least one acyl halide R¹—(C═O)X and, if R¹ and R² are different, additionally with at least one acyl halide R²—(C═O)X, where X is Br or Cl, in the presence of at least one tertiary amine to give compounds of the formula (I.3).

44. The process according to claim 43, where the leaving group Z is a moiety selected from the group consisting of Br, Cl, and the tosyl, mesyl and triflyl group.
